(19) United States
(12) Patent Application Publication
(10) Pub. No.: US 2026/0232860 A1
(43) Pub. Date: Aug. 13, 2026

(54) Mattress with antimicrobial air ventilation unit with integral LED UV light source and such an air ventilation unit

(71) Applicant: UBED B.V., Amsterdam (NL)

(72) Inventor: Frans de la Haye, Den Haag (NL)

(73) Assignee: UBED B.V., Amsterdam (NL)

(21) Appl. No.: 19/160,040

(22) PCT Filed: Mar. 4, 2024
(86) PCT No.: PCT/NL2024/050100

§ 371 Date: Aug. 27, 2025

(30) Foreign Application Priority Data
Mar. 2, 2023 (NL) 2034255

(51) Int. Cl. A61L 9/20; A47C 21/04; A47C 23/00

(52) U.S. Cl. CPC A61L 9/20; A47C 21/044; A47C 23/00; A61L 2209/12; A61L 2209/134

(57) ABSTRACT

This invention relates to a mattress with integrated antimicrobial air ventilation unit (10) for treating air with UVC light comprising:
at least one centrifugal fan unit (20) comprising an impeller (21) and a ducted fan housing (22); and
at least one UVC radiation unit (31) comprising (i) at least one UVC-LED (32) and optionally (ii) at least one UVC directing lens (34),
wherein the UVC radiation unit(s) (31) are configured to allow irradiation of air passing through the antimicrobial air ventilation unit (10),
wherein the at least one UVC radiation unit (31) is configured to illuminate at least a part of the interior of the ducted fan housing (22) with UVC light.

This invention relates to an antimicrobial air ventilation unit (10) for treating air with UVC light. Also the invention relates to method for the antimicrobial treatment of an air flow using such an antimicrobial air ventilation unit (10) for treating air with UVC light. Moreover, the invention relates to a mattress comprising such an antimicrobial air ventilation unit (10) for treating air with UVC light.

### BACKGROUND

Air contamination is a long-standing problem, which has drawn considerable attention over the years. Typically, untreated air comprises bacteria, mould spores and viruses, which can cause health problems if inhaled. This is a particular problem in interior spaces where air may be recirculated by air ventilation units, such as elevators and hospital wards. Airborne bacterial infections that may be spread via an air ventilation unit include whooping cough, diphtheria and tuberculosis. Airborne bacterial infections that may be spread via an air ventilation unit include the common cold, COVID-19, measles morbillivirus, chickenpox virus, influenza virus, enterovirus, norovirus and less commonly adenovirus. In an enclosed space, such as an office space, hospital ward or an elevator, recirculated air can increase the residence time of microbes in the air, hence the increase the probability that a susceptible person in that space will be infected by the microbe.

In the art, ventilation systems for beds are known in which an air ventilation unit is typically arranged externally adjacent to the mattress of the bed. With such air ventilated beds a same problem as described above may occur, where an air flow generated by the air ventilation system can release bacteria, mould spores, viruses or microbes.

UVC light is well known to possess a very powerful germicidal effect capable of inactivating a wide spectrum of microorganisms, such as viruses, bacteria, protozoa, fungi, yeasts, and algae, through the formation of pyrimidine dimers, the photoproducts of genetic materials. Dimerization of pyrimidine disturbs DNA replication and transcription, which may lead to cell death. However, cell death is not required to anti-microbially treat air, as long as sufficient damage to the cells has been caused so as to inhibit reproduction is required to render the microbes entrained in the air anti-microbially treated. A minimum residence time under UVC illumination is required to render microbes incapable of reproduction. This residence time is dependent on the wavelength of light, and the strength of illumination. To provide compact air ventilation units, high light intensity is required, and the state-of-the-art systems use UV bulbs with high wattage (6 W).

Such antimicrobial air conditioning systems have been provided that anti-microbially treat the air that transits through the air ventilation unit. U.S. Pat. No. 5,925,230 discloses an air purification system comprising a plurality of noisy axial fans and an ultraviolet (UV) light source (UV lamp bulb) within an air ventilation housing. One disadvantage of systems of this type is that the air flow must be brought close to the UV lamp, in this case with air being deviated around a UV lamp placed across the middle of C-shaped airflow ducting. This is bulky and causes turbulence in the airflow, and consequently causes noise. This also causes energy inefficiencies due to the turbulent flow. This prior art solution is therefore unsuitable for compact air ventilation units that rely on laminar air-flows to reduce noise and energy consumption. Until now, UV irradiation has mostly been performed with conventional low-pressure mercury UV lamps (LP lamps), which emit UV light with peak wavelength of 254 nm.

A further consideration is that UVC light is damaging to people's skin and eyes, which may cause cancer in the long term. Therefore, where UVC light sources are used in air ventilation units, care is taken to ensure that UVC light does not escape such systems. This is achieved in known systems by employing a series of baffles within the air ducting or bends within the air ducting (typically, V-, W-, U-, C- or S-shaped) to occlude light. These light occluding elements typically render such air ventilation units bulky and loud to operate, with the baffles and/or bends in the air ducting causing turbulence, which created disadvantageous noise and is energy inefficient. An example of an S-shaped air ducting assembly is seen in U.S. Pat. No. 5,925,230.

It therefore remains a challenge to provide antimicrobial air ventilation units for treating air with UVC light that operate quietly, at greater energy efficiency (lower power, in Watts) and without bulky, light-occluding elements that induce turbulent air-flow.

### BRIEF SUMMARY OF THE DISCLOSURE

In accordance with the present inventions there is provided a mattress comprising an antimicrobial air ventilation unit (10) for treating air with UVC light as defined in claim 1.

According to an aspect there is provided a method for the antimicrobial treatment of an air flow comprising the steps of:

Providing air comprising microbes to an antimicrobial air ventilation unit as defined above; Causing the air comprising microbes to flow through the centrifugal fan unit; Irradiating the air comprising microbes with UVC light flowing through the centriufugal fan unit within the ducted fan housing of the antimicrobial air ventilation unit.

### BRIEF DESCRIPTION OF THE DRAWINGS

Embodiments of the invention are further described hereinafter with reference to the accompanying drawings, in which:

FIG. 1 depicts a perspective view of a first embodiment of an antimicrobial air ventilation unit according to the invention.

FIG. 2 is a cut-away view of the first embodiment of an antimicrobial air ventilation unit according to the invention.

FIG. 3 is a horizonal, top-down, cross-section view of the first embodiment of an antimicrobial air ventilation unit according to the invention.

FIG. 4 is top-down, horizontal cross-sectional view of a non-limiting example of the first embodiment of an antimicrobial air ventilation unit according to the invention.

FIG. 5 depicts a lamb-type UVC LED suitable for use in the invention.

FIG. 6 depicts a chip-type UVC LED suitable for use in the invention.

FIG. 7 depicts a UVC radiation unit (31) comprising 9 chip-type LEDs as depicted in FIG. 6 suitable for use in the invention.

FIG. 8 depicts the light occluding nature of a light occluding baffle assembly according to the invention.

FIG. 9 depicts the non-limiting example of FIG. 4, with the dotted lines depicting direct emission paths of UVC photons from the UVC radiation units (31) being absorbed by the plates (51) of the light occluding baffle assembly (50).

FIG. 10 is a cartoon of use of an antimicrobial air ventilation unit (10) according to the first embodiment of the present invention to antimicrobially treat air.

FIG. 11 is a cartoon of use of an example of an antimicrobial air ventilation unit (10) according to the first embodiment of the present invention to antimicrobially treat air, in which the ducted fan housing (22) comprises a portion of mirrored interior surface of the ducted fan housing (22M).

FIG. 12 depicts in cartoon fashion antimicrobial treating air using a unit as depicted in FIGS. 4, 8 and 9.

FIG. 13 depicts in cartoon fashion antimicrobial treating air using a particularly preferred embodiment unit broadly corresponding to that depicted in FIGS. 4, 8 and 9, wherein the ducted fan housing (22) is mirrored.

FIG. 14 depicts in cartoon fashion antimicrobial treating air using a particularly preferred embodiment unit broadly corresponding to that depicted in FIG. 2 combined with FIG. 12.

FIG. 15 depicts in cartoon fashion antimicrobial treating air comprising microbes (depicted as rings) and dust particles (pentagons) in an embodiment according to FIG. 1

FIG. 16 depicts a particularly preferably embodiment of the present invention, in which the unit (10) additionally comprises a filter unit (80).

FIG. 17 depicts a non-limiting example corresponding to that of FIG. 9, in which the ducted fan housing (22) is mirrored.

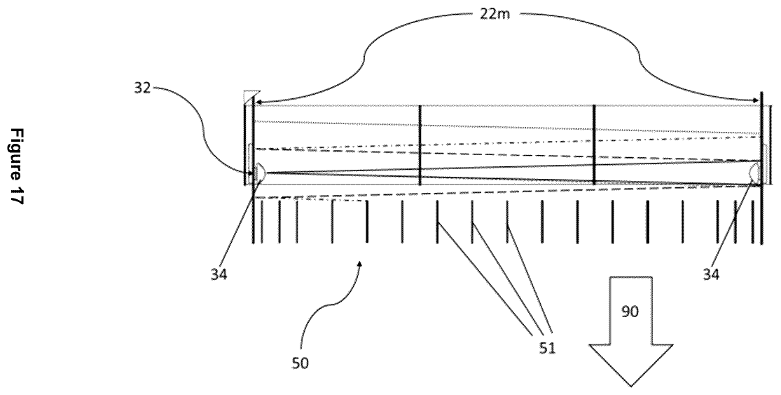

FIG. 18 schematically shows a mattress which is internally provided with an antimicrobial air ventilation unit according to an embodiment of the invention.

### DETAILED DESCRIPTION

In a first aspect, the invention concerns a mattress internally provided with an antimicrobial air ventilation unit (10) for treating air with UVC light comprising:
at least one centrifugal fan unit (20) comprising an impeller (21) and a ducted fan housing (22); and
at least one UVC radiation unit (31) comprising at least one UVC-LED (32);
wherein the at least one UVC radiation unit (31) is configured to illuminate the interior of the ducted fan housing (22) with UVC light, such that mechanical axis of the UVC-LED(s) (33) intersects the ducted fan housing (22).

The present invention achieves the goal of antimicrobially treating air with improved energy efficiency over the prior art by synergistically:
(i) moving air with greater energy efficiency;
(ii) generating photons with greater energy efficiency;
(iii) maximising the probability that a photon generated will impinge on a microbe in the moved air; and
(iv) maximising the probability that when a photon generated hits a microbe, it stops the microbe from reproducing.

A centrifugal fan unit (20) is a mechanical device for moving air or other gasses in a direction at an angle to the incoming air, typically expelling air in a direction at 90° to the incoming air. Centrifugal fan units (20) encompass fan units that may be referred to as “cross-flow fan” units (120) and “tangential fan” units (220). Centrifugal fan units (20) do not encompass fan units referred to as “axial fan” units or “axial blower” units. Centrifugal fan units (20) are not positive-displacement devices (pumps). Centrifugal fans comprise a ducted fan housing (22) and an impeller (21) (air moving means). The impeller (21) (air moving means) are typically provided by an axially symmetric rotor impeller (121) or cylindrical impeller (221), which rotate about their axes of rotation (26). The centrifugal fan unit (20) uses the centrifugal power supplied from the rotation of the impeller (21) (air moving means) to increase the kinetic energy of air or other gasses.

The impeller (21) (air moving means) may optionally be a fan wheel impeller (321) comprising an impeller hub (23) along the axis of rotation (26) and a number of impeller fan blades (24) attached to the periphery of the hub (23).

I Impeller fan blades (24) may be arranged around an impeller (21) in three different ways: (i) forward-curved/inclined impeller fan blades (24a); (ii) backward-curved/backward inclined impeller fan blades (24b) or (iii) straight radial impeller fan blades (24c).

The impeller fan blades (24) may optionally be forward-curved impeller fan blades (24b), that is the curve of the blade is configured to be in the direction of the fan wheels rotation. For small diameter fan wheels, the blade may be straight, in which case the blade is referred to as forward inclined. Antimicrobial air ventilation units (10) comprising an impeller (21), wherein the impeller (21) comprises forward-curved impeller fan blades (24b) are particularly advantageous in providing laminar air flows with minimal noise production.

The impeller fan blades (24) may optionally be backward-curved, that is the curve of the blade is against the direction of the fan wheel's rotation. For particularly small diameter fan wheels, the blade may be straight, in which case the blade is referred to as backward-inclined. Antimicrobial air ventilation units (10) comprising an impeller (21), wherein the impeller (21) comprises backward-curved or backward-inclined blades are particularly advantageously energy efficient air moving means. They are more energy efficient than comparable air ventilation units comprising forward-curved or inclined impeller fan blades (24a), but at the disadvantage of operating with slightly more noise pollution. The noise pollution generated by air ventilation units (10) comprising backward-curved or backward-inclined blades are still advantageously quieter and more energy efficient than comparable “axial fan” units or “axial blower” units. Antimicrobial air ventilation units (10) comprising an impeller (21), wherein the impeller (21) comprises backward-curved/backward inclined impeller fan blades (24b) are advantageously resistant to the solid accumulation/fouling and may be advantageously used for ventilating gasses with moderate to high particulate loadings.

The impeller fan blades (24) may optionally be straight radial impeller fan blades (24c), which extend straight out from the centre of the hub. Antimicrobial air ventilation units (10) comprising an impeller (21), wherein the impeller (21) comprises straight radial blades are the least sensitive to solid accumulation and are particularly advantageous for ventilation of airs with high to very high particulate loadings (as would be encountered with filter free operation in dusty conditions, such as in grain silos or kitchen environments).

Preferably, the impeller (21) (air moving means) is a hollow cylindrical impeller (421) with a hollow (25) aligned along the axis of rotation (26). The ends of the hollow cylindrical impeller (421) may be capped with solid end walls (27), as is typically the case for tangential fan units (220). By ends is meant an end region comprising the last 10% of the length of the impeller along the axis of rotation at either end of the impeller. The hollow cylindrical impeller (421) features impeller fan blades (24), which may be (i) forward-curved/inclined impeller fan blades (24a), (ii) backward curved/inclined impeller fan blades (24b) or (iii) straight radial impeller fan blades (24c). Dependent on the length of the hollow cylindrical impeller (421) along the axis of rotation (26), there may optionally be impeller fan blade support disks (28) to ensure impeller fan blade (24) rigidity and dimensional integrity. These impeller fan blade support disks (28) may be arranged so that their walls (29) are orthogonal to the axis of rotation (26), so that they do not impede the tangential flow of air.

In the absence of a ducted fan housing (22), when a hollow cylindrical impeller (421) or a fan wheel impeller (321) is rotated an equilibrium is created. Throughout the whole cross section of the impeller (21), air is being stirred in concentric circles with a stable vortex located in the centre of the impeller (21). In the absence of a ducted fan housing (22), minimal effective ventilation work is done, as little volume of air would transit through such a putative fan.

To produce work, air must travel through the impeller (21) (air rotation means), such as a hollow cylindrical impeller (421) or a fan wheel impeller (321). This is achieved by interaction of the impeller (21) with the ducted fan housing (22), which are configured to provide a laminar flow of air through the centrifugal fan unit (20).

In the case of an impeller (21) comprising a number of impeller fan blades (24), the ducted fan housing (22) is configured so that when the impeller fan blades (24) rotate about the axis of rotation (26), the gas particles near the impeller fan blades (24) are displaced radially outward and move towards the ducted fan housing (22). As a result, the kinetic energy of gas results in an increased pressure near the ducted fan housing (22), because of the system resistance offered by the ducted fan housing (22). The gas is then rotationally displaced within the ducted fan housing (22) to the second (outlet) side (20b) of the centrifugal fan unit, where the gas exits the centrifugal fan unit (20). As the gas is radially displaced by the impeller fan blades (24), the gas pressure near the axis of rotation decreases. The external gas from the first (inlet) side of the centrifugal fan unit (20a) rushes in to the centrifugal fan unit (20) normalize the pressure. This cycle repeats and therefore the gas can be continuously transferred through the centrifugal fan unit (20).

For tangential centrifugal fan units (220), this means that air must enter from at least one first (inlet) side of the tangential centrifugal fan unit (220a), which may beat a large range of tangential angles with respect to the second (outlet) side of the cross-flow centrifugal fan unit (220b), which is what “tangential” refers to. For tangential centrifugal fan units (220) this works by offsetting the position of the vortex from the axis of rotation to create an imbalance in pressures. Shifting the position of the vortex is accomplished by a non-axially symmetric (with respect to the axis of rotation of the impeller (21)) ducted fan housing (22a). This may be achieved by configuring the ducted fan housing (23a) so as to form an obstruction (22b) near the outer diameter of the impeller (21), which impedes rotation of gas (e.g. air) around the outside of the impeller (21).

The obstruction (22b) in the ducted fan housing (22a) is commonly known as a “vortex tongue” and its shape and position determines the performance characteristics of the tangential centrifugal fan unit (220) as well as the change in direction of the air flow. This shifting of the vortex creates high gas velocity in the centre of the impeller increasing the dynamic pressure, reducing static pressure. This creates suction on the first (inlet) side of the tangential centrifugal fan unit (220a). As the air exits through the second (outlet) side of the tangential centrifugal fan unit (220b) the gas velocity slows, decreasing the dynamic pressure and increasing static pressure. This causes the gas to exit the tangential centrifugal fan unit (220) via the second (outlet) side of the tangential centrifugal fan unit (220b). Thus, a fast, smooth laminar flow out of the tangential centrifugal fan unit (220) is established at a particularly advantageous high velocity for a given motor speed. Advantageously a laminar air flow produced by the tangential centrifugal fan unit typically exhibits a low noise rate at relatively high velocity of the air flow.

For cross-flow centrifugal fan units (120), this means that air must enter from at least one first (inlet) side of the cross-flow centrifugal fan unit (120a), which is appropriately orthogonal to the second (outlet) side of the cross-flow centrifugal fan unit (120b). This is what “cross-flow” refers to.

The size and shape of the centrifugal fan unit (20) and the size and shape fan housing are mutually dependent and are preferably configured so as to allow a laminar air flow to be formed in use.

The first aspect of the invention requires that the antimicrobial air ventilation unit (10) for treating air with UVC light comprises at least one UVC radiation unit (31) comprising (i) at least one UVC-LED (32), and optionally (ii) at least one UVC lens (34).

Suitable UVC radiation units (31) are capable of producing light with one or more wavelengths in the range of from 100 to 280 nm, more preferably in the range of from 225 to 280 nm, even more preferably in the range of from 255 to 270 nm and most preferably with a wavelength of 265 (±0.5) nm. Using a wavelength of from 100 to 280 nm maximizes the probability that when a photon generated with this wavelength hits a microbe, it stops the microbe from reproducing. The more preferable wavelength ranges are particularly advantageous as this is most destructive to bacteria and virus DNA and/or RNA, hence most efficacious at antimicrobial treatment of air.

UVC radiation units (31) comprising UVC-LEDs (32) are more energy efficient at generating photons with a wavelength of from 100 to 280 nm than conventional low-pressure mercury UV lamps (LP lamps), thereby generating photons with greater energy efficiency.

UVC radiation units (31) comprising UVC-LEDs (32) are also much faster at generating sufficient UVC radiation, doing so within seconds, compared to the minutes required by conventional low-pressure mercury UV lamps (LP lamps) to “warm up”. This provides the benefit that an antimicrobial air ventilation unit (10) according to the present invention does not needlessly circulate air for up to 15 minutes before antimicrobially treating air, and is consequently more energy efficient.

UVC radiation units (31) comprising UVC-LEDs (32) generate directionally focused light, rather than random direction of light generated by gas-discharge within a conventional mercury vapour lamp. This provides the benefit that an antimicrobial air ventilation unit (10) according to the present invention maximizes the probability that a photon generated will impinge on a microbe in the moved air, as the directionally focused light may be directed across the path through which the air is moved.

UVC radiation units (31) comprising UVC-LEDs (32) are smaller than conventional mercury vapor lamp capable of outputting the same amount of UVC radiation. The effect of this difference is that the smaller UVC radiation units (31) may be located within the ducted fan housing (22) without significantly objecting the air flow path. This results in an apparatus capable of (i) moving air with greater energy efficiency and (ii) moving air more quietly than an apparatus with a conventional mercury vapour lamp located within the ducted fan housing (22).

UVC radiation units (31) comprising UVC-LEDs (32) are believed to have a longer lifespan of approximately 25,000 hours, compared to the shorter lifespan of 5,000 hours of a typical low pressure mercury lamp. This means that an antimicrobial air ventilation unit (10) according to the present invention may be advantageously operated for longer periods than known antimicrobial UVC ventilation units without maintenance.

Suitable UVC-LEDs (32) are capable of producing light with a wavelength of 100-280 nm, more preferably in the range of from 225 to 280 nm, even more preferably in the range of from 255 to 270 nm and most preferably with a wavelength of 265 (±0.5) nm. For example, suitable UVC-LEDs may optionally be NCSU334B LED lights commercially available from Nichia (2021). UVC-LEDs are particularly advantageous in providing high flux of light with a wavelength of from 100 to 280 nm in an energy efficient manner.

Preferably, the UVC-LED(s) (32) emit light with a peak wavelength (λₚ) of from 250 to 280 nm. More preferably, the UVC-LED(s) (32) have a peak wavelength (λₚ) of from 225 to 270 nm, which is deemed particularly lethal to most microorganisms and which is herein referred to as ultraviolet germicidal irradiation (UVGI). Most preferably, the UVC-LED(s) (32) have a peak wavelength (λₚ) of 265 (±0.5) nm. This wavelength is particularly advantageous as this is most destructive to bacteria and virus DNA.

Preferably, the UVC-LED(s) (32) have a maximum irradiance flux density of from 0.5 to 50 mW/cm² at a distance of 50 mm form the UVC-LED, more preferably 1.0 to 25 mW/cm², even more preferably 1.5 to 15 mW/cm².

Preferably, the UVC radiation unit(s) (31) have a directivity angle 2θ_{1/2} of from 180° to 5°, more preferably of from 160° to 10°, even more preferably of from 140° to 20°, yet more preferably of from 120° to 30° and most preferably of from 70° to 50°. A directivity angle that is too small (smaller than) 5° is less preferable as little of the air moved though the ducted fan housing (22) may be irradiated by a single UVC-LED, leading to less energy efficiency. A directivity angle that is too large (greater than 180°) is less preferable as a significant part of the radiant flux of UVC-light hits the ducted fan housing (22) before passing through the air to be treated which is being moved through the centrifugal fan unit (20), leading to less energy efficiency.

Where the UVC radiation unit(s) (31) do not comprise at least one UVC converging lens, the UVC-LED(s) (32) preferably have a directivity angle 2θ_{1/2} of from 180° to 5°, more preferably of from 160° to 10°, even more preferably of from 140° to 20° and most preferably of from 120° to 30°. The preferable directivity angles allow for maximising the probability that a photon generated will impinge on a microbe in the moved air.

The UVC radiation unit(s) (31) preferably additionally comprise at least one UVC lens. The UVC lens is made of a material that is optically transmissive to light with a wavelength in the range of from 100 to 280 nm that is resistant to photodegradation at this wavelength. Suitable materials include UV-resistant plastics and quartz glass. The function of the UVC lens(es) (34) is to modify the directivity angle 2θ_{1/2} of the light emitted by the UVC-LED(s) (32). The UVC lens(es) (34) and UVC-LED(s) (32) may be configured such that the UVC radiation emitted by each UVC-LED impinges a UVC lens (34), which results in an overall change in the directivity angle 2θ1/2. The UVC lens (34) may be a converging lens or a diverging lens, more preferably a converging lens. Particularly preferable UVC radiation unit(s) (31) comprise a combination of a converging UVC lens and a UVC-LED (32), to afford UVC radiation unit(s) (31) with a directivity angle 2θ_{1/2} of from 180° to 5°, more preferably of from 160° to 10°, even more preferably of from 140° to 20°, yet more preferably of from 120° to 30° and most preferably of from 70° to 50°. Preferably, the UVC radiation unit(s) (31) is (are) configured to illuminate the interior of the ducted fan housing (22) with UVC light, such that optical axis (axes) of the UVC radiation unit(s) (31) is (are) approximately co-axial with the axis of rotation of the impeller (26). By approximately co-axial, it is meant that the axes are within 25° of co-axial. An advantage of this configuration is that this allows light from the UVC radiation unit(s) (31) to travel along the length of the ducted fan housing (22L), approximately orthogonal (within 25° of orthogonal) to the flow of air. This allows the UVC photons to travel for a maximal distance though microbe-containing air, which increases the probability that any one photon of UVC light will impinge on a microbe travelling through the ducted fan housing. The effect of this is that lower irradiance flux, and consequently less energy is required to achieve the same degree of antimicrobial treatment. This arrangement is described in this patent application as enfilading UVC illumination.

More preferably, the UVC radiation unit(s) (31) is (are) configured to illuminate the interior of the ducted fan housing (22) with UVC light, such that the optical axis(es) of the UVC radiation unit(s) (31) is (are) approximately co-axial with the axis of rotation of the impeller (26), wherein by approximately co-axial, it is meant that the axes are within 15° of co-axial, even more preferably it is meant that the axes are within 10° of co-axial, most preferably it is meant that the axes are within 5° of co-axial.

Most preferably, the UVC radiation unit(s) (31) is (are) configured to illuminate the interior of the ducted fan housing (22) with UVC light, such that all air flowing through the antimicrobial air ventilation unit (10) is exposed to UVC radiation.

In a preferable embodiment, the UVC radiation unit(s) (31) is (are) configured to illuminate the interior of the ducted fan housing (22) with UVC light, such that mechanical axis (axes) of the UVC-LED(s) (33) is (are) approximately co-axial with the axis of rotation of the impeller (26). By approximately co-axial, it is meant that the axes are within 25° of co-axial. An advantage of this configuration is that this allows light from the UVC-LED to travel along the length of the ducted fan housing (22L), which increases the probability that any one photon of UVC light will impinge on a microbe travelling through the ducted fan housing and consequently lower irradiance flux and less energy is required to achieve the antimicrobial treatment. This could be considered to be enfilading UVC illumination of a laminar air flow.

More preferably, the UVC radiation unit (31) is configured to illuminate the interior of the ducted fan housing (22) with UVC light, such that mechanical axis of the UVC-LED(s) (33) is approximately co-axial with the axis of rotation of the impeller (26), wherein by approximately co-axial, it is meant that the axes are within 15° of co-axial, even more preferably it is meant that the axes are within 10° of co-axial, most preferably it is meant that the axes are within 5° of co-axial.

Most preferably, the UVC radiation unit (31) is configured to illuminate the interior of the ducted fan housing (22) with UVC light, such that all air flowing through the antimicrobial air ventilation unit (10) is exposed to UVC radiation.

In a preferable embodiment, the antimicrobial air ventilation unit (10) for treating air with UVC light comprises:
at least one centrifugal fan unit (20) comprising an impeller (21) and a ducted fan housing (22); and
at least two UVC radiation units (31), each UVC radiation unit comprising at least one UVC-LED (32);
wherein the at least two UVC radiation units (31) are configured to illuminate the interior of the ducted fan housing (22) with UVC light, such that mechanical axis of the UVC-LED(s) (33) intersects the ducted fan housing (22).

Preferably, the at least two UVC radiation units (31) are located on or at opposite sides of the ducted fan housing (22) and are configured to simultaneously illuminate a volume of the ducted fan housing (22K). This simultaneously illuminated volume of the ducted fan housing is termed the enfilading microbial kill-zone of the ducted fan housing (22K). The advantage of this configuration is that higher radiant flux of UVC light can be achieved for low energy costs compared to other configurations, which in use allow high energy efficiency antimicrobial treatment of air moved through the ducted fan housing (22).

Most preferably, the at least two UVC radiation units (21) are located on or at opposite sides of the ducted fan housing (22), are configured to simultaneously illuminate a volume of the ducted fan housing (22K) and are configured such that mechanical axes of the UVC-LED(s) (33) are approximately co-axial with the axis of rotation of the impeller (26). By approximately co-axial, it is meant that the axes are within 45° of co-axial. An advantage of this configuration is that this allows light from the UVC-LED to travel along the length of the ducted fan housing (22L), from two directions, which increases the probability that any one photon of UVC light will impinge on a microbe travelling through the ducted fan housing and consequently lower irradiance flux and less energy is required to achieve the antimicrobial treatment. This means that higher UVC photon flux can be achieved in the enfilading microbial kill-zone of the ducted fan housing (22K), and consequently higher flow rates can be realised without comprising the antimicrobial treatment. Even more preferably, the UVC radiation units (31) are configured to illuminate the interior of the ducted fan housing (22) with UVC light, such that mechanical axes of the UVC-LEDs (33) are approximately co-axial with the axis of rotation of the impeller (26), wherein by approximately co-axial, it is meant that the axes are within 30° of co-axial, yet more preferably it is meant that the axes are within 10° of co-axial, most preferably it is meant that the axes are within 5° of co-axial. The closer to co-axial the axes of the UVC-LEDs (33) are to the axis of rotation of the impeller, the higher the flux of UVC photons in the enfilading microbial kill-zone of the ducted fan housing (22K) can be achieved for the same UVC-LEDs (32).

In an equally preferable alternative embodiment, the antimicrobial air ventilation unit (10) for treating air with UVC light comprises:
at least one centrifugal fan unit (20) comprising an impeller (21) and a ducted fan housing (22);
at least one UVC radiation unit (31) comprising at least one UVC-LED (32); and
at least one mirrored interior surface of the ducted fan housing (22M),
   wherein the at least one UVC radiation unit (31) is configured to illuminate the interior of the ducted fan housing (22) with UVC light, such that mechanical axis of the UVC-LED(s) (33) intersects the mirrored interior surface of the ducted fan housing (22M).

This embodiment advantageously allows higher UVC photon flux to be achieved within the ducted fan housing (22) than a comparable antimicrobial air ventilation unit without at least one mirrored interior surface of the ducted fan housing (22M) or configured such that mechanical axis of the UVC-LED(s) (33) intersects the mirrored interior surface of the ducted fan housing (22M).

By mirrored interior surface of the ducted fan housing (22M) is meant a surface that reflects at least 15% of incident light radiation with a wavelength of from 100 to 280 nm. A suitable mirrored interior surface would be stainless steel sheet, which reflects approximately 20-30% of incident UVC light with a wavelength of 100-280 nm, or untreated aluminium, which reflects approximately 40-60% of incident UVC light with a wavelength of 100-280 nm, or chromium plating, which reflects approximately 39% of incident UVC light with a wavelength of 100-280 nm, or aluminium paint, which reflects approximately 10-75% of incident UVC light with a wavelength of 100-280 nm. Preferably, the mirrored interior surface of the ducted fan housing (22M) reflects at least 70% of incident light radiation with a wavelength of 100-280 nm. Such a surface could suitably be made of aluminium foil, which reflects approximately 73% of incident light radiation with a wavelength of 100-280 nm, or magnesium oxide which reflects approximately 75-88% of incident light radiation with a wavelength of 100-280 nm. More preferably the mirrored interior surface of the ducted fan housing (22M) reflects at least 75% of incident light radiation with a wavelength of 100-280 nm. Such a surface could suitably be made of polished aluminium sheet (alzak), which reflects approximately 80% of incident light radiation with a wavelength of 100-280 nm. Even more preferably the mirrored interior surface of the ducted fan housing (22M) reflects at least 90% of incident light radiation with a wavelength of 100-280 nm. Such a surface could suitably be made of expanded Polytetrafluoroethylene (e-PTFE), which reflects approximately 95% of incident light radiation with a wavelength of 100-280 nm.

Preferably, the at least one UVC radiation unit (31) and at least one mirrored interior surface of the ducted fan housing (22M) are located on or at opposite sides of the ducted fan housing (22) and are configured such that un-reflected light from the at least one UVC radiation unit (31) and reflected light from the mirrored interior surface of the ducted fan housing (22M) simultaneously illuminate a volume within the ducted fan housing (22K). This simultaneously illuminated volume of the ducted fan housing is termed the enfilading microbial kill-zone (reflected) of the ducted fan housing (22KR). The advantage of this configuration is that higher radiant flux of UVC light can be achieved for low energy costs compared to other configurations, which in use allow high energy efficiency antimicrobial treatment of air moved through the ducted fan housing (22), in particular air moved through the enfilading microbial kill-zone (reflected) of the ducted fan housing (22

More preferably, the at least one UVC radiation units (31) and at least one mirrored interior surface of the ducted fan housing (22M) are configured to simultaneously illuminate a volume of the ducted fan housing (22KR) and are configured such that mechanical axis (axes) of the UVC-LED(s) (33) is (are) approximately co-axial with the axis of rotation of the impeller (26) and substantially orthogonal to the at least one mirrored interior surface of the ducted fan housing (22M). By approximately co-axial, it is meant that the axes are within 45° of co-axial. By substantially orthogonal, it is meant that the axis is within 20° of normally incident. An advantage of this configuration is that this allows light from the UVC-LED to travel along the length of the ducted fan housing (22L), from two directions, which increases the probability that any one photon of UVC light will impinge on a microbe travelling through the ducted fan housing and consequently lower irradiance flux and less energy is required to achieve the antimicrobial treatment. This means that higher UVC photon flux can be achieved in the enfilading microbial kill-zone (reflected) of the ducted fan housing (22KR), and consequently higher flow rates can be realised without comprising the antimicrobial treatment.

Even more preferably, the at least one UVC radiation unit (31) is configured to illuminate the interior of the ducted fan housing (22) with UVC light, such that mechanical axis (axes) of the UVC-LED(s) (33) is (are) approximately co-axial with the axis of rotation of the impeller (26), wherein by approximately co-axial, it is meant that the axes are within 30° of co-axial, yet more preferably it is meant that the axes are within 10° of co-axial, most preferably it is meant that the axes are within 5° of co-axial. The closer to co-axial the axis (axes) of the UVC-LED(s) (33) is (are) to the axis of rotation of the impeller, the higher the flux of UVC photons in the enfilading microbial kill-zone (reflected) of the ducted fan housing (22KR) can be achieved for the same number of UVC-LED(s) (32) in alternative configurations.

Even more preferably, the at least one UVC radiation unit (31) is configured to illuminate the interior of the ducted fan housing (22) with UVC light, such that mechanical axis (axes) of the UVC-LED(s) (33) is (are) substantially orthogonal to the at least one mirrored interior surface of the ducted fan housing (22M), wherein by substantially orthogonal is meant that the axis is (axes are) within 10° of normally incident, yet more preferably within 5° of normally incident and most preferably are within 2° of normally incident.

A favourable combination of features is an the antimicrobial air ventilation unit according to this embodiment wherein:
the mirrored interior surface of the ducted fan housing (22M) reflects at least 20% of incident light radiation with a wavelength of 100-280 nm;
1 mechanical axis (axes) of the UVC-LED(s) (33) is (are) approximately co-axial with the axis of rotation of the impeller (26), wherein approximately co-axial means that the axes are within 45° of co-axial; and
mechanical axis (axes) of the UVC-LED(s) (33) is (are) substantially orthogonal to the at least one mirrored interior surface of the ducted fan housing (22M), wherein substantially orthogonal means that the axis is within 20° of normally incident.

Another, particularly favourable combination of features is an the antimicrobial air ventilation unit according to this embodiment wherein:
the mirrored interior surface of the ducted fan housing (22M) reflects at least 70% of incident light radiation with a wavelength of 100-280 nm;
mechanical axis (axes) of the UVC-LED(s) (33) is (are) approximately co-axial with the axis of rotation of the impeller (26), wherein approximately co-axial means that the axes are within 30° of co-axial; and
mechanical axis (axes) of the UVC-LED(s) (33) is (are) substantially orthogonal to the at least one mirrored interior surface of the ducted fan housing (22M), wherein substantially orthogonal means that the axis is within 10° of normally incident.

Another, more particularly favourable combination of features is an the antimicrobial air ventilation unit according to this embodiment wherein:
the mirrored interior surface of the ducted fan housing (22M) reflects at least 75% of incident light radiation with a wavelength of 100-280 nm;
mechanical axis (axes) of the UVC-LED(s) (33) is (are) approximately co-axial with the axis of rotation of the impeller (26), wherein approximately co-axial means that the axes are within 5° of co-axial; and
mechanical axis (axes) of the UVC-LED(s) (33) is (are) substantially orthogonal to the at least one mirrored interior surface of the ducted fan housing (22M), wherein substantially orthogonal means that the axis is within 10° of normally incident.

Another, even more particularly favourable combination of features is an the antimicrobial air ventilation unit according to this embodiment wherein:
the mirrored interior surface of the ducted fan housing (22M) reflects at least 80% of incident light radiation with a wavelength of 100-280 nm;
mechanical axis (axes) of the UVC-LED(s) (33) is (are) approximately co-axial with the axis of rotation of the impeller (26), wherein approximately co-axial means that the axes are within 10° of co-axial; and
mechanical axis (axes) of the UVC-LED(s) (33) is (are) substantially orthogonal to the at least one mirrored interior surface of the ducted fan housing (22M), wherein substantially orthogonal means that the axis is within 5° of normally incident.

Another, most favorable combination of features is an antimicrobial air ventilation unit according to this embodiment wherein:
the mirrored interior surface of the ducted fan housing (22M) reflects at least 90% of incident light radiation with a wavelength of 100-280 nm;
mechanical axis (axes) of the UVC-LED(s) (33) is (are) approximately co-axial with the axis of rotation of the impeller (26), wherein approximately co-axial means that the axes are within 5° of co-axial; and
mechanical axis (axes) of the UVC-LED(s) (33) is (are) substantially orthogonal to the at least one mirrored interior surface of the ducted fan housing (22M), wherein substantially orthogonal means that the axis is within 2° of normally incident.

The more the incident light radiation with a wavelength of 100-280 nm the mirrored interior surface of the ducted fan housing (22M) reflects at least 90% of, the higher the flux of UVC photons in the enfilading microbial kill-zone (reflected) of the ducted fan housing (22KR) can be achieved than for alternative configurations utilizing less reflective mirrored interior surface of the ducted fan housings (22M).

The closer to co-axial the axis (axes) of the UVC-LED(s) (33) is (are) to the axis of rotation of the impeller, the higher the flux of UVC photons in the enfilading microbial kill-zone (reflected) of the ducted fan housing (22KR) can be achieved than for alternative configurations utilizing the same UVC-LED(s) (32).

The closer to normally incident the axis (axes) of the UVC-LED(s) (33) is (are) to the at least one mirrored interior surface of the ducted fan housing (22M), the higher the flux of UVC photons in the enfilading microbial kill-zone (reflected) of the ducted fan housing (22KR) can be achieved than for alternative configurations utilizing the same UVC-LED(s) (32).

The antimicrobial air ventilation unit (10) according to the first aspect of the invention, according to any of the embodiments above, may optionally additionally comprise an air ventilation housing (40).

The air ventilation housing (40) is preferably configured to enclose the at least one centrifugal fan unit (20) and the UVC radiation unit (31), whilst still allowing fluid communication of air from the outside of the antimicrobial air ventilation unit (10) to the first (inlet) side of the at least one centrifugal fan unit (20a) and the second (outlet) side of the at least one centrifugal fan unit (20). The air ventilation housing (40) advantageously protects the antimicrobial air ventilation unit (10) from accidental damage and external environmental factors.

The antimicrobial air ventilation unit (10) according to the first aspect of the invention, according to any of the embodiments above, may optionally additionally comprise a light occluding baffle assembly (50) comprising a plurality of light occluding baffle assembly slits (51).

Where present, the light occluding baffle assembly (50) is configured to fit the second (outlet) side of the at least one centrifugal fan unit (20b). The light occluding baffle assembly advantageously protects the centrifugal fan unit from accidental ingress of large objects into the ducted fan housing (22).

In a preferable embodiment, the light occluding baffle assembly (50) comprises:
a plurality of light occluding baffle assembly slits (51); and
a plurality of light occluding baffle assembly plates (52),
   wherein the plurality of light occluding baffle assembly slits (51) and the plurality of light occluding baffle assembly plates (52) are configured and oriented to visibly occlude the UVC radiation unit(s) (31) whilst still allowing free fluid communication of air from the interior of the centrifugal fan unit (20) with the exterior of the antimicrobial air ventilation unit (10) via the second (outlet) side of the at least one centrifugal fan unit (20). Visibly occluding the UVC radiation unit(s) (31) means that no UVC light emitted by the UVC radiation units (31) has a direct path to the exterior of the antimicrobial air ventilation unit (10).

A suitable light occluding baffle assembly (50) so configured could be a light occluding baffle assembly (50) wherein the plurality of light occluding baffle assembly plates (52) are arranged to be substantially orthogonal to the axis of rotation of impeller (26) [and consequently substantially parallel to direction of air flow out of the second (outlet) side of the at least one centrifugal fan unit (20)].

An effect of this embodiment is that little to no UVC-light exits the antimicrobial air ventilation unit (10), which confers the advantage of being safer (less to no harmful radiation exposure). Another effect is that this obviates the need for additional light occluding elements, such as a series of baffles within the air ducting or bends within the air ducting (typically, V-, W-, U-, C- or S-shaped). These light occluding elements typically render such air ventilation units bulky and loud to operate, thus this advantageously allows for the provision of more compact antimicrobial air ventilation units.

Preferably, the plurality of light occluding baffle assembly plates (52) are coated in a material that reflects less than 30% of incident light radiation with a wavelength of 100-280 nm. A suitable surface would be polished steel, which reflects approximately 20-28% of incident UVC light with a wavelength of 100-280 nm. More preferably, the plurality of light occluding baffle assembly plates (52) are coated in a material that reflects less than 10% of incident light radiation with a wavelength of 100-280 nm. Even more preferably, the plurality of light occluding baffle assembly plates (52) are coated in a material that reflects less than 2% of incident light radiation with a wavelength of 100-280 nm. Most preferably, the plurality of light occluding baffle assembly plates (52) are coated in a material that reflects less than 2% of incident light radiation with a wavelength of 100-280 nm.

The less that the plurality of light occluding baffle assembly plates (52) reflect incident light radiation with a wavelength of 100-280 nm, the less potentially harmful UVC light that may escape the antimicrobial air ventilation unit (10) by reflecting off the light occluding baffle assembly plates (52), and hence safer the antimicrobial air ventilation unit (10) is in use.

The antimicrobial air ventilation unit (10) according to the first aspect of the invention, according to any of the embodiments above, may optionally additionally comprise a filtration unit (80). The optional filtration unit (80) is preferably proximal to the first (inlet) side of the at least one centrifugal fan unit (20a). The filtration unit (80) may be selected from a high-efficiency particulate air (HEPA) filter (80a).

The HEPA filter (80a) is preferably configured to remove at least 99.95% of particles whose diameter is equal to 0.3 μm from the air that passes through the HEPA filter, measured according to method described in European Standard EN 1822-1:2019, and references cited therein.

A second aspect of the invention concerns a method for the antimicrobial treatment of air comprising the steps of:
i. Providing air comprising microbes to an antimicrobial air ventilation unit (10) according to any of the embodiments according to the first aspect of the invention;
ii. Causing the air comprising microbes to move through the centrifugal fan unit (20); and
iii. Irradiating the moving air comprising microbes with UVC light within the ducted fan housing (22) of the antimicrobial air ventilation unit (10).

In the context of the current invention antimicrobial treatment is a reduction in the active microorganism concentration. This may be quantified by an inactivation rate, which is a measurement in the reduction in active microorganism concentration is expressed as N₀/N (%) or log (N₀/N), in which:
N₀ is the original active microorganism concentration, and
N is the active microorganism concentration after microbial treatment.

Another useful parameter is the fractional kill after time t, which is defined as 1−Nₜ/N₀, wherein:
N₀ is the original active microorganism concentration; and
Nₜ is the active microorganism concentration after time t.

The method according to the second aspect of the invention is advantageous in that it provides almost instantaneous UVC radiation time compared to methods that employ conventional mercury lamps which typically require a warm up time of up to 15 minutes.

Preferably, the method is a method for the antimicrobial treatment of air, wherein the microbes are selected from fungal spores, bacterial spores, mycobacteria, vegetative bacteria and viruses. More preferably, the method is a method for the antimicrobial treatment of air, wherein the microbes are selected from bacterial spores, mycobacteria, vegetative bacteria and viruses. Even more preferably, the method is a method for the antimicrobial treatment of air, wherein the microbes are selected from mycobacteria, vegetative bacteria and viruses. Yet more preferably, the method is a method for the antimicrobial treatment of air, wherein the microbes are selected vegetative bacteria and viruses. Most preferably, the method is a method for the antiviral treatment of air.

Preferably, the method is one wherein the air is moved through the centrifugal fan unit (20) at a rate of from [values] m³/s.

Preferably, the method comprises the additional step of:

(iv) filtering the air comprising microbes using a filter unit (80) before causing the air comprising microbes to move through the centrifugal fan unit (20)

A third aspect of the invention relates to a method for the antimicrobial treatment of air comprising the steps of:
i. providing air comprising microbes to an antimicrobial air ventilation unit according to any of the embodiments above;
ii. causing the air comprising microbes to move through the centrifugal fan unit;
iii. irradiating the moving air comprising microbes with UVC light within the ducted fan housing of the antimicrobial air ventilation unit.

A fourth aspect of the invention relates to the use of the antimicrobial air ventilation unit (10) according to any of embodiments according to the first aspect in controlling the air quality of an internal space, preferably for controlling the air quality of a room or vehicle cabin space, most preferably a room.

A fifth aspect of the invention relates to an air-conditioning unit comprising an antimicrobial air ventilation unit (10) according to any of embodiments according to the first aspect.

A sixth aspect of the invention relates to a mattress comprising an antimicrobial air ventilation unit (10) according to any of embodiments according to the first aspect.

A seventh aspect of the invention relates to an elevator comprising an antimicrobial air ventilation unit (10) according to any of embodiments according to the first aspect.

### Definitions

In the context of the current invention, the following definitions are used:

UVC: ultraviolet C, light with a wavelength of 100-280 nm. This usage is in accordance with ISO standard ISO-21348:2007.

LED: A light-emitting diode (LED) is a semiconductor light source that emits light when current flows through it.

Irradiance flux density is the radiant flux (power) received by a surface per unit area. The SI unit of irradiance is the watt per square meter (W·m⁻²). A point source of light produces spherical wave-fronts, in which case irradiance flux density varies inversely with the square of the distance from the source.

The directivity angle indicates the range the light emitted from a LED radiates, expressed in degrees. Directivity is determined by viewing the change in light output when rotating the package, measuring from the output peak value up to what angle the light is still visible. Numerically, since directivity is normally symmetrical and covers the left and right sides when viewed from the front, it is indicated by twice the angle (±) at which the light output is half the maximum output, denoted 2θ_{1/2}.

LED front tip: the LED front tip is the center point of the LED light emitting surface on the outer surface of the emitter.

LED optical axis. This is the axis through the LED emitter front tip in the direction of the centroid of the optical radiation pattern.

LED peak intensity axis. This is the axis through the LED emitter front tip in the direction of the maximum intensity.

LED mechanical axis. This is the axis through the LED emitter front tip in the direction of the axis of symmetry of the emitter body for lamb-type LEDs. For chip-type LEDs the mechanical axis corresponds to the axis about which the lens element (i) has the highest rotational symmetry, and (ii) if two or more axes possess the same rotational symmetry, then the axis with the highest mirror symmetry is selected.

Peak wavelength (λₚ) is defined as the single wavelength where the radiometric emission spectrum of the light source reaches its maximum.

Normally incident to a surface is at 90° with respect to the surface.

An optical axis is a line along which there is some degree of rotational symmetry in an optical system.

UV susceptibility is the extent to which a microorganism is sensitive to UVC light or how easily it can be inactivated by UV irradiation. UV susceptibility depends on the species and character of the microorganism. It can be described by a UV susceptibility constant (k) with the unit of m²/J.

UV dose (D) is the product of UV irradiance and specific exposure time on a given microorganism (expressed in millijoules per square centimetre, mJ/cm²). The longer the time a microbe is exposed to UV light, the higher the UV dose it will receive. In a device with evenly distributed UV irradiation and airflow, the UV dose can be calculated based on the definition above.

Average UV dose (AD). In the current invention, the average UV dose is determined by the inactivation rate and a known microbial susceptibility.

Inactivation rate: reduction in active microorganism concentration is expressed as N₀/N (%) or log (N₀/N),

in which:
N₀ is the original active microorganism concentration, and
N is the active microorganism concentration after antimicrobial treatment.

UV dose-response curve is quantified relationship between the inactivation rate of a specific microorganism and the average UV dose (AD) it received.

The relationship typically follows the equation as below:

$$In ( {N}_{0} / N ) = kAD \qquad ( 1 )$$

in which AD, k and In(N₀/N) are as described above. In Formula (1), N/N₀ or AD can be calculated with the other parameters known. In other cases, the relationship may not strictly follow Formula (1), but N/N₀ or AD can also be determined according to the specific curve.

ISO 15714: 2019 (en) “Method of evaluating the UV dose to airborne microorganisms transiting in-duct ultraviolet germicidal irradiation devices” provides the method by which this inactivation rate may be determined from the UV dose-response curve.

### DESCRIPTION OF THE EMBODIMENTS

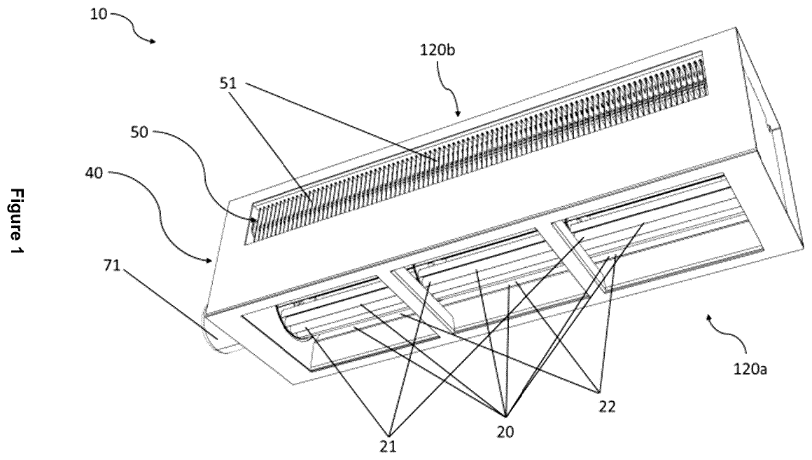

FIG. 1 depicts a perspective view of a first embodiment of an antimicrobial air ventilation unit (10) according to the invention. In the embodiment depicted here, the antimicrobial air ventilation unit (10) is suitable for treating air with UVC light. The antimicrobial air ventilation unit (10) comprises a centrifugal fan unit (20), which itself comprises an impeller (21) and a ducted fan housing (22). In the embodiment depicted in FIG. 1, the antimicrobial air ventilation unit (10) comprises at least one UVC radiation unit (31), which is configured to reside within the antimicrobial air ventilation unit (10) and thus not depicted. The UVC radiation unit(s) (31) comprise at least one UVC-LED (32). The UVC radiation unit(s) may optionally additionally comprise at least one UVC directing lens. Such a UVC directing lens may optionally be used to obtain a source of UVC light with a narrower directivity angle 2θ_{1/2} than from a commercially available UVC-LED. This optional UVC directing lens can be advantageous used in producing a higher flux of UVC light across a flow of air comprising microbes, with a resultant higher energy efficiency for antimicrobially treating air with the antimicrobial air ventilation unit (10). The UVC radiation units (31) are configured to allow irradiation of air passing through the antimicrobial air unit (10). The at least one UVC radiation unit (31) is configured to illuminate at least a part of the interior of the ducted fan housing with UVC light.

FIG. 1 additionally depicts optional features of the present invention. The depicted embodiment may optionally feature a light occluding baffle assembly (50). The optional light occluding baffle assembly is configured to allow air to flow the baffle assembly and to occlude direct rays of UVC light emitted by the UVC LED unit(s) (31). The optional light occluding baffle assembly (50) therefore prevents direct emission of the UVC light generated by the UVC LED unit(s) to the exterior of the antimicrobial air ventilation unit (10). In a particularly preferred configuration, the light occluding baffle assembly (50) is configured to allow air to pass through without generating more than 40 decibels of sound, more preferably less than 30 decibels of sound, even more preferably less than 20 decibels of sound, most preferably less than 10 decibels of sound. In a particularly preferable configuration, the light occluding baffle assembly (50) comprises a series of light occluding baffle plates, where the plates are preferably substantially orthogonal to the rotational axis (axes) of the impeller(s) (26). By substantially orthogonal is means that the axis is within 20° of normally incident. In an even more preferably configuration, the light occluding baffle assembly (50) comprises a series of light occluding baffle plates, where the plates are substantially orthogonal to the rotational axis (axes) of the impeller(s) (26), wherein by substantially orthogonal is meant that the axis is (axes are) within 10° of normally incident, yet more preferably within 5° of normally incident and most preferably are within 2° of normally incident.

FIG. 1 further depicts the optional feature of an air ventilation housing (40). FIG. 1 additionally depicts the optional feature of an integrated motor within a motor housing (71). The means of moving the impeller may also be externally provided to the antimicrobial air ventilation unit (10), such as by a chain drive or gear mechanism. The advantage of incorporating an optional motor within the antimicrobial air ventilation unit (10) is that no external moving means is required, making installation of a complete unit easier.

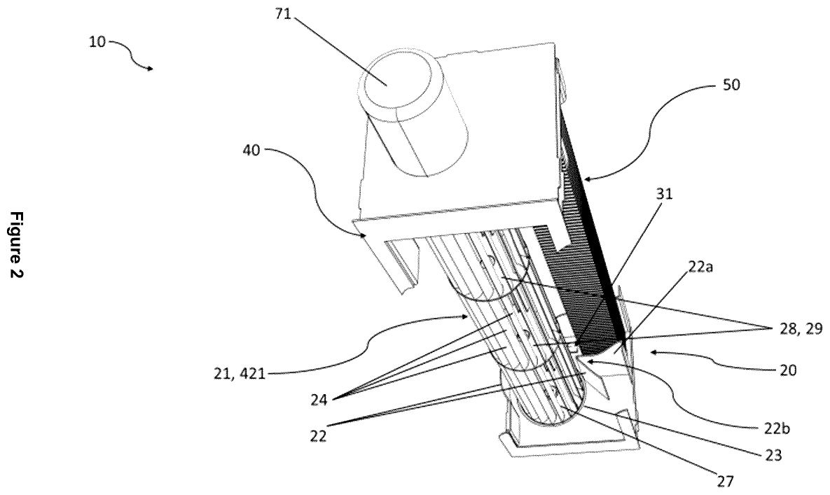

FIG. 2 depicts an antimicrobial air ventilation unit (10) according to an embodiment of the present invention in a cut away view, with both the optional air ventilation housing (40) and essential ducted fan housing (22) partially cut away. By way of a non-limiting example, the centrifugal fan unit (20) comprises a tangential cross-flow centrifugal fan. By way of non-limiting example, the tangential cross-flow centrifugal fan comprises a hollow cylindrical impeller (421). For this non-limiting example, the tangential cross-flow centrifugal fan is located within the ducted fan housing (22), and the ducted fan housing (22) has been configured so as to form an obstruction (22b) near the outer diameter of the impeller (21), which impedes rotation of gas (e.g., air) around the outside of the impeller (21, 421). The obstruction (22b) proximal to the hollow cylindrical impeller (421) is typically called a “vortex tongue”. For tangential cross-flow centrifugal fans, the ducted fan housing (22) needs to be axially asymmetric (not-axially symmetric) with respect to the axis of rotation of the impeller.

In this non-limiting example, the hollow cylindrical impeller (421) features two solid end walls (27) delimited radially in the impeller hubs (23), with the impeller fan blades (24) attached to the solid end walls (27) proximal to the impeller hubs (23). In this example, the hollow cylindrical impeller (421) further features two optional impeller fan blade support disks (28), which are attached to the impeller fan blades (24), and each impeller fan blade support disk (28) comprises an impeller fan blade support disk wall (29). The optional feature of impeller fan blade support disks is conferring additional structural rigidity and hence may beneficially extend operational lifetime. By way of non-limiting example, the blades are arranged with rotational symmetry around the rotational axis of the impeller. The skilled person will readily appreciate that minor deviation from rotational symmetry, such as by omitting a blade or minor structural modification of a blade, will not diminish the efficacy of the impeller. In this particular example, the blades of the impeller (24) are configured to occlude direct rays of UVC light emitted by the UVC LED unit(s) (31) passing through the impeller (21). This feature of the blades being configured in a light occluding configuration is not limiting, and the skilled person can readily envisage alternative configurations. One advantage of a light occluding configuration of the impeller blades (24) is that UVC light is not emitted from the antimicrobial air ventilation unit (10) through the impeller (21), obviating any need for a further light occluding baffle assembly. The embodiment is depicted with the optional feature of a light occluding baffle assembly (50). By way of non-limiting example, the light occluding baffle assembly (50) may consist of parallel plates (51), which are configured substantially orthogonal to the axis of rotation of the impeller and oriented relative to the light path from the LED unit to block light from the UVC LED unit(s) from exiting the air ventilation unit. FIG. 2 additionally depicts the optional features of an integrated motor within a motor housing (71) and an air ventilation housing (40).

FIG. 2 depicts a UVC radiation unit (31) comprising a UVC-LED (32). By way of non-limiting example, it is attached to an interior wall of the antimicrobial air ventilation unit (10) and is configured to emit UVC radiation substantially parallel to the rotational axis of the impeller (21). In this non-limiting example, the UVC radiation unit (31) is configured to illuminate a space defined by the (i) the ducted fan housing (22) and (ii) between the impeller (21) and (iii) the light occluding baffle assembly (50) as described above, in particular in the space “down-wind” from the impeller when the antimicrobial air ventilation unit (10) is in use. This confers the advantage that most UVC light generated cannot directly exit the antimicrobial air ventilation unit (10), obviating the need for any further light occluding winds/turns in the ducted fan housing. This beneficially results in a more compact antimicrobial air ventilation unit (10), which operates with less noise pollution from air turbulence. The skilled person will readily appreciate that the UVC radiation unit (31) may be configured to illuminate a space defined by the (i) the ducted fan housing (22) and (ii) between the impeller (21) and (iii) the light occluding baffle assembly (50) as described above, either “up-wind” or “down-wind” from the impeller when the antimicrobial air ventilation unit (10) is in use, with substantially the same result.

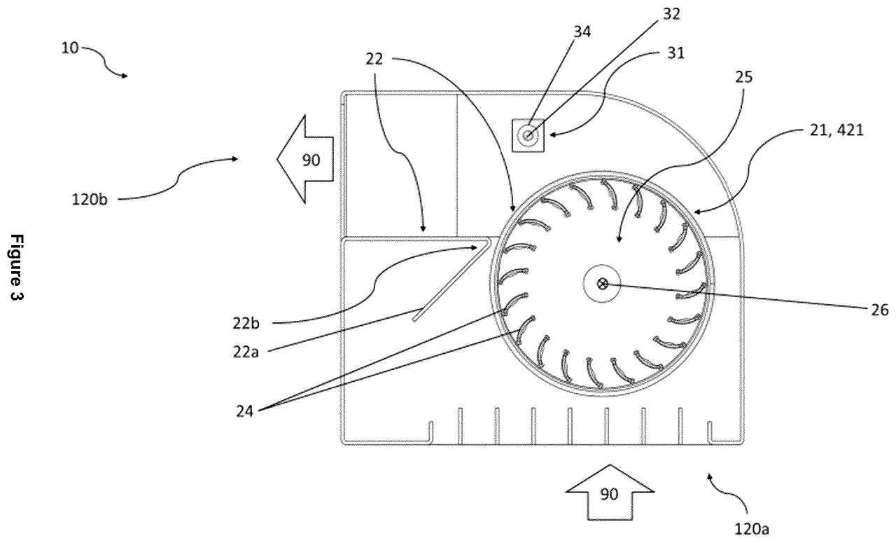

FIG. 3 depicts a vertical cross-section view of the first embodiment of an antimicrobial air ventilation unit according to the invention. In this non-limiting example, a cross section of the hollow cylindrical impeller (421) depicts the impeller fan blades (24) as curved fan blades arranged in a rotationally symmetric manner around the rotational axis of the impeller (26). The hollow space (25) defined by the by end walls (not depicted, 27) and the impeller fan blades (24) is rotationally symmetric around the rotational axis of the impeller (26). By way of non-limiting example, the UVC radiation unit (31) in this example comprises both (i) an essential UVC LED (32) and (ii) an optional UVC directing lens (34).

When in use, the air comprising microbes enters the antimicrobial air ventilation unit (10) from below (vertical arrow, 90). The air is moved through the unit (10) by rotation of the impeller (21, 421) around its axis of rotation (26). The impeller (21), in conjunction with the “vortex junction” constituted by the obstruction (22b) causes a substantially laminar flow of air through/past the impeller substantially orthogonal to the rotational axis of the impeller. This is advantageously quiet compared to rotary fan units known in the art for moving air in ventilation systems. As the air exits the impeller, a substantial fraction of the laminar air flow passes through a volume illuminated by UVC radiation emitted by the UVC radiation unit (31). For each photon of UVC light that impinges on a microbe within the moving laminar air column, there is a probability that the UVC will to the microbe so as to inhibit the microbe from reproducing. The antimicrobial treated laminar air column then exits the unit (10), as depicted by the horizontal arrow (90). Optionally, the air may pass through a light obstructing baffle assembly (i) prior to passing through/past the impeller (21) and/or (ii) after passing through/past the impeller (21).

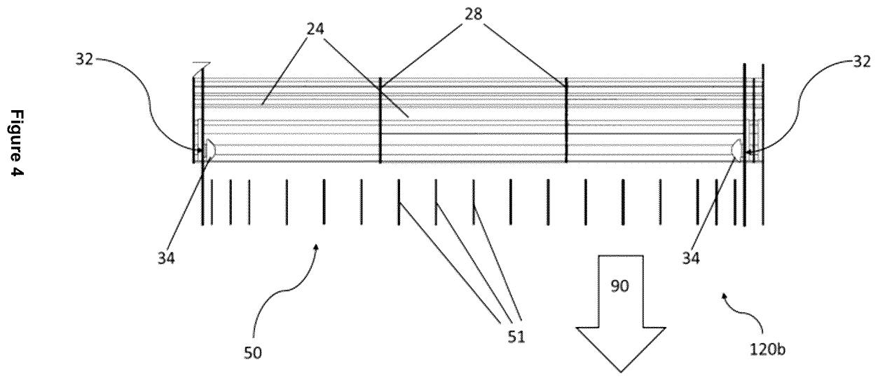

FIG. 4 is a top-down, horizonal cross-sectional view of a non-limiting example of the first embodiment of an antimicrobial air ventilation unit (10) according to the invention. In this example, two UVC radiation units (31), each comprising a UVC LED (32) and a UVC directing lens (34), are located on opposite faces of the ducted fan housing (22). By way of non-limiting example, the optical axes of both UVC radiation units are substantially parallel to the rotational axis of the impeller (not depicted, 26). The two UVC radiation units (31) are configured to allow illumination of substantially all of the air passed through the unit (10) during use, allowing more rapid antimicrobial treatment of an enclosed space ventilated by the unit (10), such as an elevator or hospital room. One possible configuration of a light occluding baffle assembly (50) is depicted, comprising a set of substantially parallel plates (51), which are configured substantially orthogonal to the rotational axis of the impeller (21). When in use, the air comprising microbes exits the impeller (21) and substantially all of the laminar air flow passes through a volume illuminated by UVC radiation emitted by the two UVC radiation units (not labelled here, 31). For each photon of UVC light that impinges on a microbe within the moving laminar air column, there is a probability that the UVC will to the microbe so as to inhibit the microbe from reproducing. The antimicrobial treated laminar air column then exits the unit (10), as depicted by the arrow (90).

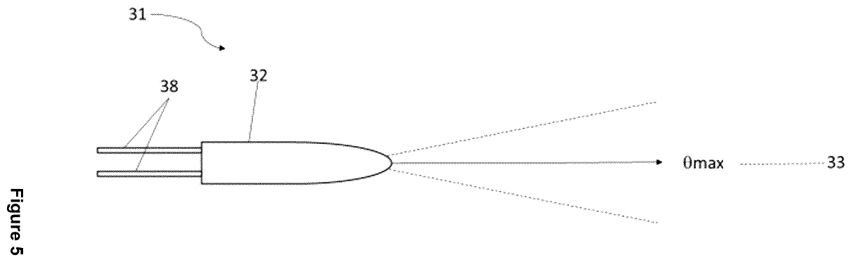

FIGS. 5-7 depict three representative types of suitable UVC radiation units. FIG. 5 depicts a UVC radiation unit (31) consisting of a typical lamb-type UVC-LED. The unit comprises two electrically conductive wires (38) and the LED emitter body (32). The mechanical axis of the LED (33) is the axis through the LED emitter body front tip (right hand side of 32) in the direction of the axis of symmetry (in this depiction an infinite rotational axis, Co) of the emitter body for lamb-type LEDs. The mechanical axis of the LED (33) is depicted as coincident with the optical axis/peak intensity axis (θ_{MAX}). The peak intensity axis is the axis corresponding to the maximum light output at a given distance from the LED emitter body (32). Lamb type LEDs typically exhibit minor deviations of the peak intensity axis (θ_{MAX}) from the mechanical axis in the range of 0-15°. FIG. 5 also depicts the first angle at which the light output is half the maximum output (36) (with respect to the peak intensity axis, θ_{MAX}) and the second angle at which the light output is half the maximum output (37) (with respect to the peak intensity axis, θ_{MAX}). The angle from the peak intensity axis, θ_{MAX}) to depicts the first angle at which the light output is half the maximum output (36) (with respect to the peak intensity axis, θ_{MAX}) defines θ. Numerically, since directivity is normally symmetrical and covers the left and right sides when viewed from the front, it is indicated by twice the angle (±) at which the light output is half the maximum output, denoted 2θ_{1/2}.

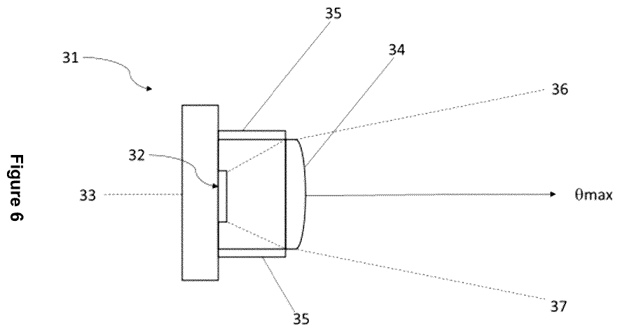

FIG. 6 depicts a UVC radiation unit (31) consisting of a chip-type LED. The chip-type LED typically comprises an LED emitter body (32) attached to a circuit board. In this instance, the LED emitter body (32) is housed in a housing (35). Attached to the housing (35) is a lens (34). The lens (34) may be suitably selected from glass or plastic resistant to UVC-radiation degradation. The mechanical axis of the LED (33) is the axis about which the lens element (34) has the highest rotational symmetry (in this depiction an infinite rotational axis, Co). The mechanical axis of the LED (33) is depicted as coincident with the optical axis/peak intensity axis (θ_{MAX}). The peak intensity axis is the axis corresponding to the maximum light output at a given distance from the LED emitter body (32). FIG. 6 also depicts the first angle at which the light output is half the maximum output (36) (with respect to the peak intensity axis, θ_{MAX}) and the second angle at which the light output is half the maximum output (with respect to the peak intensity axis, θ_{MAX}). The angle from the peak intensity axis, θ_{MAX} too the first angle at which the light output is half the maximum output (36) (with respect to the peak intensity axis, θ_{MAX}) defines θ. Numerically, since directivity is normally symmetrical and covers the left and right sides when viewed from the front, it is indicated by twice the angle (±) at which the light output is half the maximum output, denoted 2θ_{1/2}. The UVC radiation unit (31) is configured such that, when in use, UVC radiation is emitted by the light emitter body (32), which is then focused by the lens (34) to give a 2θ_{1/2} value in the range of 0.05 to 10°.

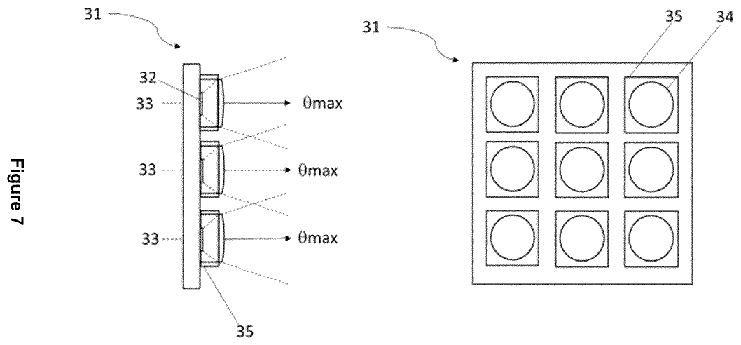

FIG. 7 depicts a UVC radiation unit (31) consisting of 9 chip-type LEDs as depicted in FIG. 6. This is a non-limiting representative example of a UVC radiation unit (31) comprising a plurality of either (i) lamb-type LEDs, (ii) chip-type LEDs or (iii) a mixture of lamb-type LEDs and chip-type LEDs. The left-hand side of FIG. 7 depicts a cross-section of the UVC radiation unit (31). The right-hand side depicts a front-on perspective of the UVC unit (31).

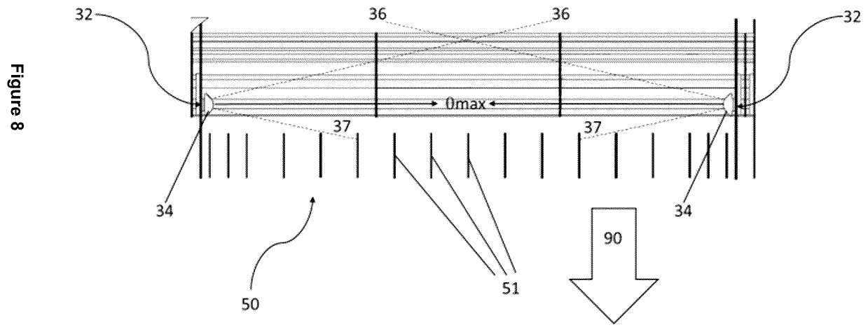

FIG. 8 corresponds to FIG. 4, with the optical axis of the two UVC radiation units (not labelled, 31, comprising 32 and 34) depicted as the arrow “θ_{MAX}” and the 2θ_{1/2} cone for each UVC radiation unit depicted between the first angle at which the light output is half the maximum output (36) (with respect to the peak intensity axis, θ_{MAX}) and the second angle at which the light output is half the maximum output (37) (with respect to the peak intensity axis, θ_{MAX}).

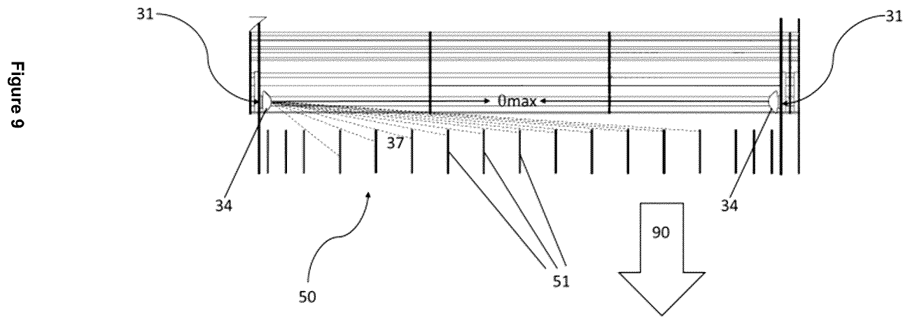

FIG. 9 corresponds to FIG. 4, with the dotted lines depicting direct emission paths of UVC photons from the UVC radiation units (31) being absorbed by the plates (51) of the light occluding baffle assembly (50).

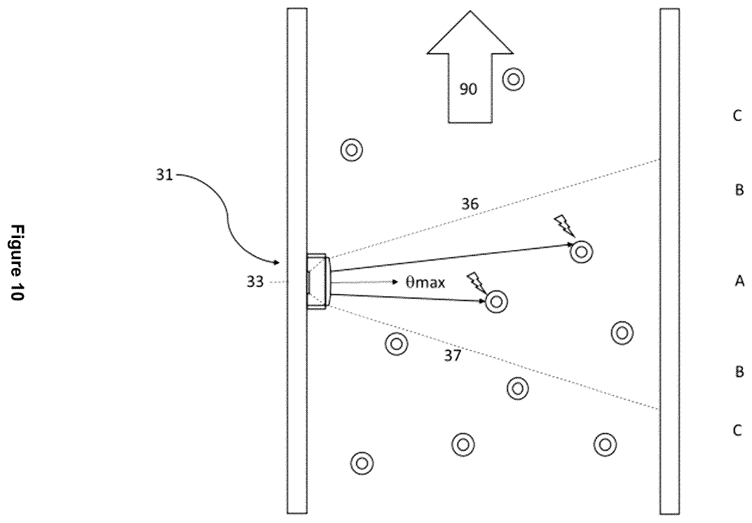

FIG. 10 is a cartoon of use of an antimicrobial air ventilation unit (10) according to the first embodiment of the present invention to antimicrobially treat air. Air comprising microbes, depicted in cartoon form as rings, enters a part of the unit (10) with a UVC radiation unit (31) attached to the ducted fan housing (22). The direction of the air flow is from bottom to top, as denoted by arrow (90). As the air comprising microbes passes through the volume illuminated by UVC radiation emitted by the UVC radiation unit (31), photons of UVC light impinge at least some of the microbes present in the air column. For each photon of UVC light that impinges on a microbe within the moving laminar air column, there is a probability that the UVC will to the microbe so as to inhibit the microbe from reproducing. Destruction of the microbe is depicted by the cartoon “lightning strike”. The antimicrobially treated laminar air column then exits the unit (10), as depicted by the horizontal arrow (90), which comprises a lower concentration of microbes.

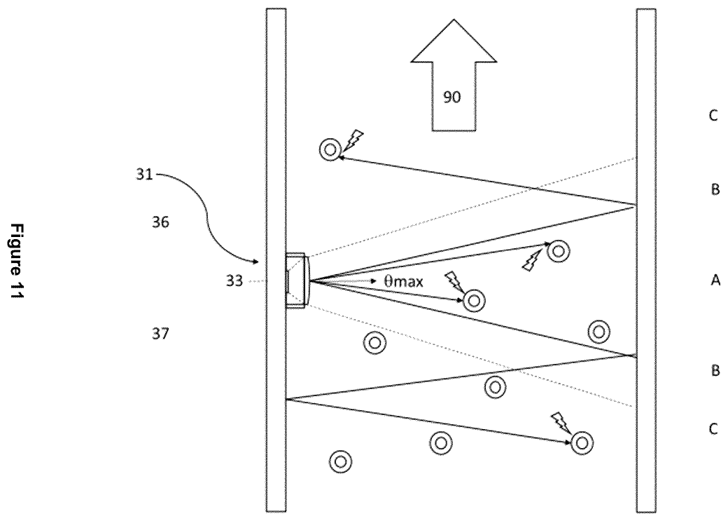

FIG. 11 broadly corresponds to FIG. 10, but ducted fan housing (22) comprises a portion of mirrored interior surface of the ducted fan housing (22M), which is located such that at least some UVC light from at least one UVC radiation unit (31) may be incident with the mirrored interior surface of the ducted fan housing (22M) during use. By way of non-limiting example. By way of non-limiting example, the mirrored interior surface of the ducted fan housing (22M) may be configured to be substantially orthogonal to the optical axis of at least one UVC radiation unit (31). Such a configuration allows a substantial portion of the incident UVC photons to be reflected, increasing their path length and increasing the probability that any one UVC photon will impinge on a microbe. This increases the probably that any one microbe will be stuck by a UVC photon and thereby inhibited from reproducing. An additional benefit of such a configuration is that substantially all air passing though such a unit (10) may be antimicrobial treated using only one UVC radiation unit.

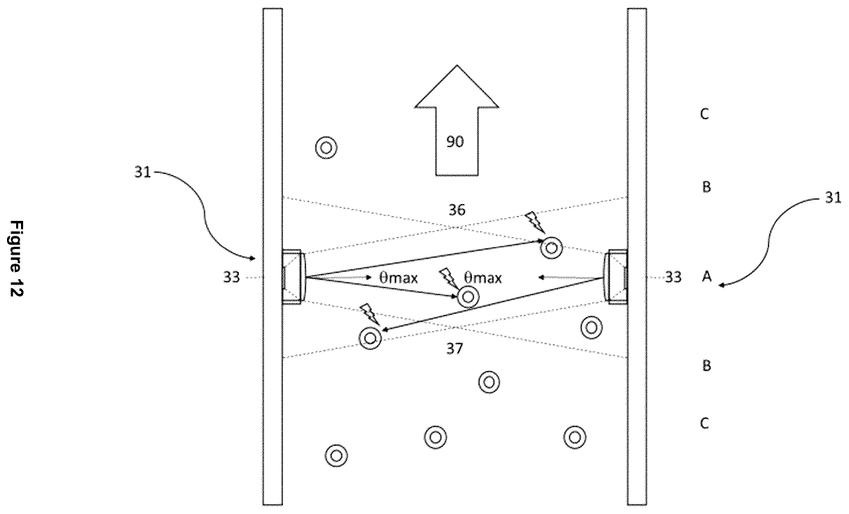

FIG. 12 depicts in cartoon fashion antimicrobial treating air using a unit as depicted in FIGS. 4, 8 and 9.

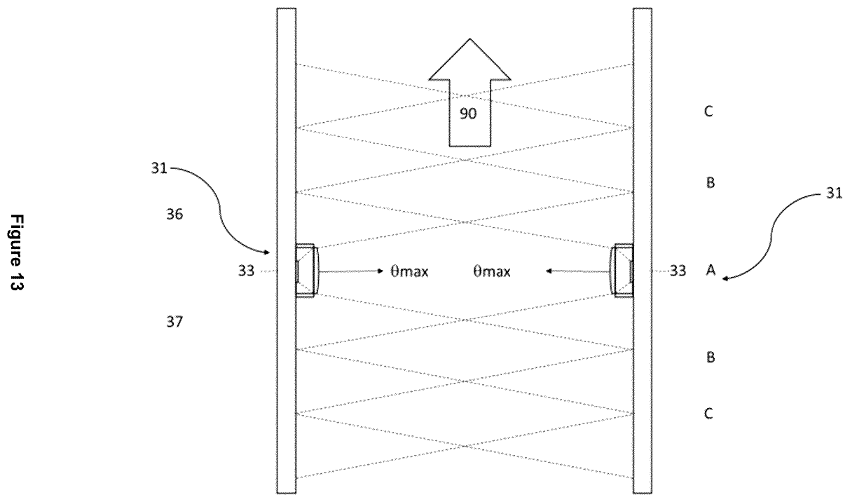

FIG. 13 depicts in cartoon fashion antimicrobial treating air using a particularly preferred embodiment unit broadly corresponding to that depicted in FIGS. 4, 8 and 9, wherein the ducted fan housing (22) is mirrored. Such a configuration allows a substantial portion of the UVC photons incident on the ducted fan housing (22) to be reflected, increasing their path length and increasing the probability that any one UVC photon will impinge on a microbe. This increases the probably that any one microbe will be stuck by a UVC photon and thereby inhibited from reproducing.

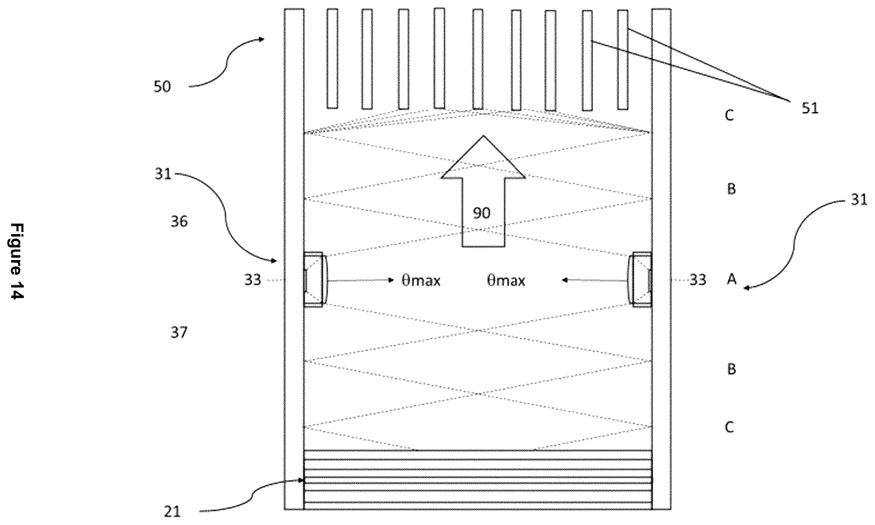

FIG. 14 depicts in cartoon fashion antimicrobial treating air using a particularly preferred embodiment unit broadly corresponding to that depicted in FIG. 2 combined with FIG. 12. Such a configuration allows a substantial portion of the UVC photons incident on the ducted fan housing (22) to be reflected, increasing their path length and increasing the probability that any one UVC photon will impinge on a microbe. This increases the probably that any one microbe will be stuck by a UVC photon and thereby inhibited from reproducing. This particularly favored embodiment confers the advantage that most UVC light generated cannot directly exit the antimicrobial air ventilation unit (10), obviating the need for any further light occluding winds/turns in the ducted fan housing. This beneficially results in a more compact antimicrobial air ventilation unit (10), which operates with less noise pollution from air turbulence and maximal energy efficiency.

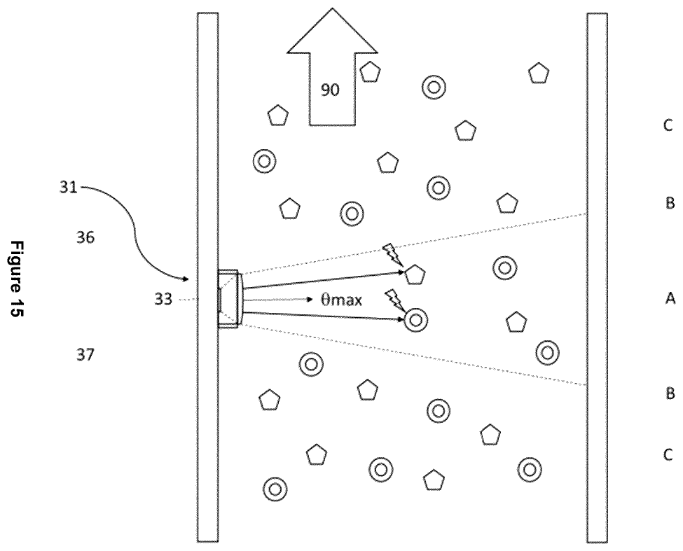

FIG. 15 depicts in cartoon fashion antimicrobial treating air comprising microbes (depicted as rings) and dust particles (pentagons) in an embodiment according to FIG. 1. UVC photons impinging dust particles may be absorbed, lowering the probability that any one UVC photon will impinge with a microbe. This reduces the probably that any one microbe will be stuck by a UVC photon and thereby inhibited from reproducing. The more dust is present, the less energy efficient the antimicrobial treatment is.

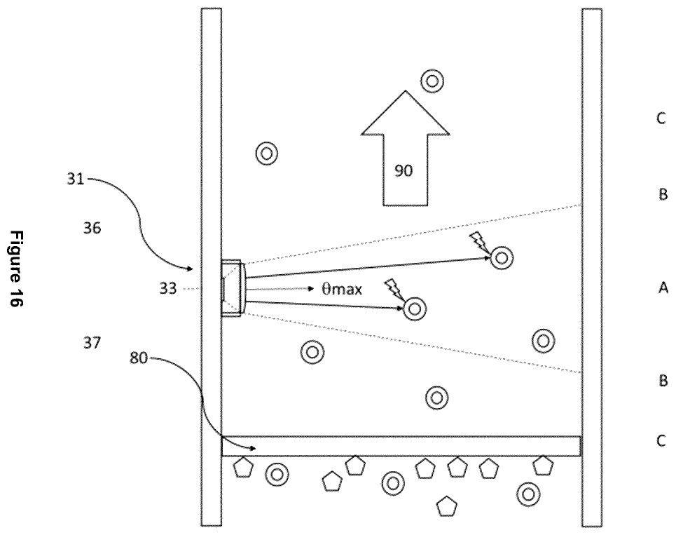

FIG. 16 depicts a particularly preferably embodiment of the present invention, in which the unit (10) additionally comprises a filter unit (80). Preferably, the filter unit is a high-efficiency particulate air (HEPA) filter (80a). The figure depicts, in cartoon form, the effect of the filter (80) in removing dust particles (pentagons) from the air to be microbially treated, thereby increasing the probability that any one UVC photon will impinge a microbe and damage it sufficiently to inhibit its reproduction. Thereby, the probability that any one microbe is inhibited from reproduction is increased and the efficiency of the apparatus for antimicrobial treating air thereby increased.

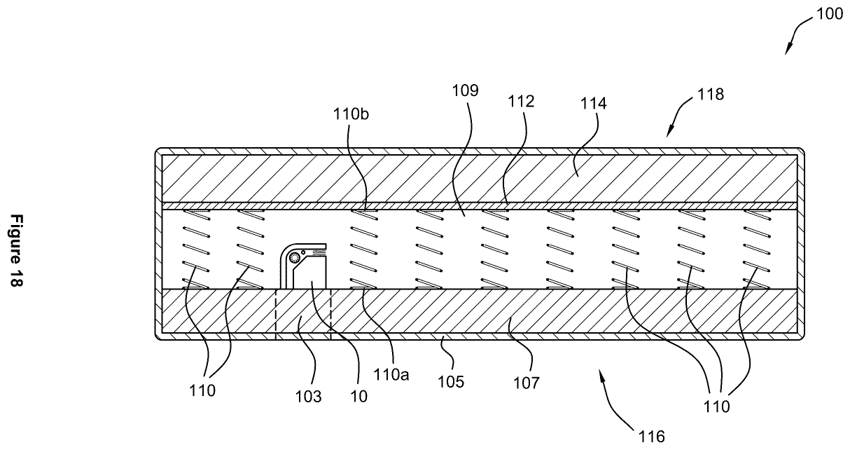

FIG. 18 shows a cross-section of a mattress 100 provided internally with an antimicrobial air ventilation unit in accordance with an embodiment of the invention. The drawing of FIG. 18 is only schematic and not on scale.

Within a body of the mattress 100 an antimicrobial air ventilation unit 10 as described above with reference to the preceding figures is arranged. The mattress has a substantially rectangular structure created by a stack of multiple parallel layers 105, 107, 109, 112, 114, each of the layers having a predetermined respective permeability for air.

The mattress 100 has a substantially all-sided cover layer 105 consisting of a fabric, which envelopes the other layers 107, 109, 112, 114 of the stack.

At a bottom surface 116 of the mattress a lower support layer 107 is arranged on the all-sided cover layer 105.

On the lower support layer 107 a carrier layer 109 is arranged which is substantially an empty volume between sidewalls of the mattress, the lower support layer 107 and an upper support layer 112 of the mattress. In the carrier layer 109 a plurality of springs 110, preferably steel Bonnell springs, is deployed with the foot 110a of each spring positioned on the lower support layer 107. In addition, the antimicrobial air ventilation unit 10 as described above is positioned in between the springs 110.

On a head 110b of each spring 110 in the carrier layer 109, i.e., the end of the spring facing away from the lower support layer 107, the upper support layer 112 is seated. At a side of the upper support layer 112 facing away from the carrier layer 109, a second upper support layer or comfort layer 114 is arranged. Finally, the second upper support layer 114 is covered at a side thereof facing way from the upper support layer 112 by the all-sided cover layer 105.

The antimicrobial air ventilation unit 10 has an entry for air that is in fluid communication with air at the bottom side 116 of the mattress by means of an intake opening for air 103. Alternatively, the mattress has a lower support layer that is permeable for air, in a manner that the antimicrobial air ventilation unit receives air through the lower support layer.

The exit for air from the antimicrobial air ventilation unit 10 debouches in the carrier layer 109 which is essentially an empty space only lodging the plurality of springs 110. As a result the carrier layer 109 has a low air resistance which allows that air pressure is substantially constant over the volume of the carrier layer 109.

The flow path of the air through the mattress is determined by the permeability for air of each of the multiple layers in the stack. The permeability for air of the upper support layer 112 and the second upper support layer 114 is relatively larger than the permeability of the lower support layer 107. The lower support layer 107 has a lower permeability that practically reduces air flow through that layer 107. The all-sided cover layer 105, the upper support layer 112 and second upper support layer 114 comprise of a material(s) that have a permeability that allows air flow through those layers 112, 114. This arrangement of layers with different resistance to air flowing through each layer provides that air from the antimicrobial air ventilation unit flows through the mattress such that air expelled from the exit of the antimicrobial air ventilation unit permeates the layers depending on their permeability and substantially exits the mattress from the top surface 118, i.e., the surface in use configured for a person to ly on. The arrangement of the multiple layers in the stack is configured such that the outflow of the air is distributed substantially evenly over the top surface.

The antimicrobial air ventilation unit is configured to draw air from the intake opening 103 in the mattress, typically located at the bottom surface 116 opposite the top surface 118. The air flows from the intake opening 103 to the entry of the air ventilation unit 10. As mentioned above, alternatively the intake opening 103 may be omitted and the air is drawn in through the lower support layer 107.

Within the antimicrobial air ventilation unit, the drawn-in air is exposed to UVC radiation generated by the UVC led(s) within the antimicrobial air ventilation unit. After UVC exposure of the air in the antimicrobial air ventilation unit, the air exiting the air ventilation unit will flow through the mattress's upper layers 112, 114, towards the top surface 118.

In addition to exposure to UV radiation, the air ventilation unit may heat the air passing through the air ventilation unit by an integrated heating unit. The heating unit is typically controlled by a temperature control unit (not shown) which comprises and is connected to one or more temperature sensors (not shown) in the mattress. In an embodiment the temperature sensors are positioned in or near the top surface 118 of the mattress.

The skilled in the art will appreciate that the mattress may contain additional layers such as a felt layer for support of the feet of the springs 110a, and a side wall layer forming the side walls surrounding a circumference of the carrier layer 109 between the lower support layer 107 and the upper support layer 112 which consists of an air-tight or low permeability layer to prevent air leakage at said circumference.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

List of reference numerals
Similar reference numbers used in the description to indicate similar elements
(but only differences in hundreds) are implicitly included.

| | |
|---|---|
| 10 | Antimicrobial air ventilation unit. |
| 20 | Centrifugal fan unit. |
| 20a | First (inlet) side of the at least one centrifugal fan unit (20). |
| 20b | Second (outlet) side of the at least one centrifugal fan unit (20). |
| 21 | Impeller. |
| 21L | Length of impeller. |
| 21D | Diameter of impeller. |
| 22 | Ducted fan housing. |
| 22a | Non-axially symmetric ducted fan housing, with respect to the axis of rotation |
| | impeller (26). |
| 22b | Deformation of the ducted fan housing (22a). |
| 22H | Height of the ducted fan housing (22). |
| 22K | Enfilading microbial kill-zone of the ducted fan housing (22). |
| 22KR | Enfilading microbial kill-zone (reflected) of the ducted fan housing (22). |
| 22L | Length of the ducted fan housing (22). |
| 22M | Mirrored interior surface of the ducted fan housing (22). |
| 22W | Width of the ducted fan housing (22). |
| 23 | Impeller hubs. |
| 24 | Impeller fan blades. |
| 24a | Forward-curved/inclined impeller fan blades. |
| 24b | Backward-curved/backward inclined impeller fan blades. |
| 24c | Straight radial impeller fan blades. |
| 25 | Hollow space. |
| 26 | Axis of rotation of impeller (21, 121, 221, 321). |
| 27 | Solid end walls. |
| 28 | Impeller fan blade support disks. |
| 29 | Impeller fan blade support disk walls. |
| 31 | UVC radiation unit. |
| 32 | UVC-LED(s). |
| 33 | Mechanical axis of the LED(s). |
| 34 | UVC directing lens. |
| 35 | LED housing. |
| 36 | First angle at which the light output is half the maximum output. |
| 37 | Second angle at which the light output is half the maximum output. |
| 40 | Air ventilation housing. |
| 50 | Light occluding baffle assembly. |
| 51 | Light occluding baffle assembly slits. |
| 52 | Light occluding baffle assembly plates. |
| 80 | Filter unit. |
| 80a | High-efficiency particulate air (HEPA) filter. |
| 90 | Direction of air flow. |
| 100 | Mattress |
| 103 | air intake opening |
| 105 | all sided cover layer |
| 107 | lower support layer |
| 109 | carrier layer |
| 110 | spring |
| 110a | foot of spring |
| 110b | head of spring |
| 112 | upper support layer |
| 114 | second support layer |
| 116 | bottom surface |
| 118 | top surface |
| 120 | cross-flow centrifugal fan unit |
| 120a | first (inlet) side of the cross-flow centrifugal fan unit (120). |
| 120b | second (outlet) side of the cross-flow centrifugal fan unit (120). |
| 121 | axially symmetric rotor impeller. |
| 220 | tangential centrifugal fan unit. |
| 220a | first (inlet) side of the tangential centrifugal fan unit (200). |
| 220b | second (outlet) side of the tangential centrifugal fan unit (200). |
| 221 | cylindrical impeller. |
| 321 | fan wheel impeller. |
| 421 | hollow cylindrical impeller. |

Additionally, various aspects of the present invention are defined by the following clauses.

clause 1: An antimicrobial air ventilation unit (10) for treating air with UVC light comprising:
at least one centrifugal fan unit (20) comprising an impeller (21) and a ducted fan housing (22); and
at least one UVC radiation unit (31) comprising (i) at least one UVC-LED (32) and optionally (ii) at least one UVC directing lens (34),
wherein the UVC radiation unit(s) (31) are configured to allow irradiation of air passing through the antimicrobial air ventilation unit (10),
wherein the at least one UVC radiation unit (31) is configured to illuminate at least a part of the interior of the ducted fan housing (22) with UVC light.

clause 2: The antimicrobial air ventilation unit (10) according to clause 1, wherein the at least one centrifugal fan unit (20) is selected from a cross-flow centrifugal fan unit (120) or a tangential centrifugal fan unit (220), more preferably the at least one centrifugal fan unit (20) is a tangential centrifugal fan unit (220).

Clause 3: The antimicrobial air ventilation unit (10) according to clause 1 or 2, wherein the impeller (21) is an axially symmetric rotor impeller (121), a cylindrical impeller (221), a fan wheel impeller (321) and/or a hollow cylindrical impeller (421).

Clause 4: The antimicrobial air ventilation unit (10) according to any preceding clause, wherein the impeller (21) is a fan wheel impeller (321), preferably wherein the fan wheel impeller (321) comprises impeller fan blades (24) selected from forward-curved/inclined impeller fan blades (24a), backward-curved/backward inclined impeller fan blades (24b) or straight radial impeller fan blades (24c), more preferably wherein the fan wheel impeller (321) comprises forward-curved/inclined impeller fan blades (24a), most preferably wherein the fan wheel impeller (321) comprises forward-curved impeller fan blades (24a).

Clause 5: The antimicrobial air ventilation unit (10) according to any preceding clause, wherein the impeller (21) is a hollow cylindrical impeller (421), preferably wherein hollow cylindrical impeller (421) comprises impeller fan blades (24) selected from forward-curved/inclined impeller fan blades (24a), backward-curved/backward inclined impeller fan blades (24b) or straight radial impeller fan blades (24c), more preferably wherein the hollow cylindrical impeller (421) comprises forward-curved/inclined impeller fan blades (24a), most preferably wherein the hollow cylindrical impeller (421) comprises forward-curved impeller fan blades (24a).

Clause 6: The antimicrobial air ventilation unit (10) according to clause 5, wherein the at least one centrifugal fan unit (20) is a tangential centrifugal fan unit (220), the impeller (21) is a hollow cylindrical impeller (421) and the ends of the hollow cylindrical impeller (421) are capped with solid end walls (27).

Clause 7: The antimicrobial air ventilation unit (10) according to any preceding clauses, wherein the UVC radiation units (31) are capable of producing light with one or more wavelengths in the range of from 100 to 280 nm, more preferably in the range of from 225 to 280 nm, even more preferably in the range of from 255 to 270 nm and most preferably with a wavelength of 265 (±0.5) nm.

Clause 8: The antimicrobial air ventilation unit (10) according to any preceding clauses, wherein the UVC-LEDs (32) are capable of producing light with a wavelength of 100-280 nm, more preferably in the range of from 225 to 280 nm, even more preferably in the range of from 255 to 270 nm and most preferably with a wavelength of 265 (±0.5) nm.

Clause 9: The antimicrobial air ventilation unit (10) according to any preceding clauses, wherein the UVC-LED(s) (32) emit light with a peak wavelength (AP) of from 250 to 280 nm, more preferably, the UVC-LED(s) (32) have a peak wavelength (AP) of from 225 to 270 nm, most preferably, the UVC-LED(s) (32) have a peak wavelength (AP) of 265 (±0.5) nm.

Clause 10: The antimicrobial air ventilation unit (10) according to any preceding clauses, wherein the UVC-LED(s) (32) have a maximum irradiance flux density of from 0.5 to 50 mW/cm² at a distance of 50 mm form the UVC-LED, more preferably 1.0 to 25 mW/cm², even more preferably 1.5 to 15 mW/cm².

Clause 11: The antimicrobial air ventilation unit (10) according to any preceding clauses, wherein the UVC-LED(s) (32) have a directivity angle 2θ_{1/2} of from 180° to 5°, more preferably of from 160° to 10°, even more preferably of from 140° to 20° and most preferably of from 120° to 30°.

Clause 12: The antimicrobial air ventilation unit (10) according to any preceding clauses, wherein the UVC radiation unit (31) is configured to illuminate the interior of the ducted fan housing (22) with UVC light, such that mechanical axis of the UVC-LED(s) (33) is approximately co-axial with the axis of rotation of the impeller (26), wherein by approximately co-axial, it is meant that the axes are within 45° of co-axial, preferably wherein it is meant that the axes are within 30° of co-axial, even more preferably it is meant that the axes are within 10° of co-axial, most preferably it is meant that the axes are within 5° of co-axial.

Clause 13: The antimicrobial air ventilation unit (10) according to any preceding clauses, wherein the antimicrobial air ventilation unit (10) comprises:
at least one centrifugal fan unit (20) comprising an impeller (21) and a ducted fan housing (22); and
at least two UVC radiation units (31), each UVC radiation unit comprising at least one UVC-LED (32);
   wherein the at least two UVC radiation units (31) are configured to illuminate the interior of the ducted fan housing (22) with UVC light, such that mechanical axis of the UVC-LED(s) (33) intersects the ducted fan housing (22).

Clause 14: The antimicrobial air ventilation unit (10) according to any previous clause, wherein the antimicrobial air ventilation unit (10) additionally comprises a light occluding baffle assembly.

Clause 15: The antimicrobial air ventilation unit (10) according to any of clauses 1-12, wherein the antimicrobial air ventilation unit (10) for treating air with UVC light comprises:
at least one centrifugal fan unit (20) comprising an impeller (21) and a ducted fan housing (22);
at least one UVC radiation unit (31) comprising at least one UVC-LED (32); and
at least one mirrored interior surface of the ducted fan housing (22M) wherein the at least one UVC radiation unit (31) is configured to illuminate the interior of the ducted fan housing (22) with UVC light, such that mechanical axis of the UVC-LED(s) (33) intersects the mirrored interior surface of the ducted fan housing (22M).

Clause 16: A method for the antimicrobial treatment of air comprising the steps of: Providing air comprising microbes to an antimicrobial air ventilation unit according to any of clauses 1-15;

Causing the air comprising microbes to move through the centrifugal fan unit; Irradiating the moving air comprising microbes with UVC light within the ducted fan housing of the antimicrobial air ventilation unit.

Clause 17: Use of the apparatus according to any of clauses 1-15 in controlling the air quality of an internal space, preferably for controlling the air quality of a room or vehicle cabin space, most preferably a room.

Clause 18: An air-conditioning unit comprising an apparatus according to any of clauses 1-15.

Clause 19: A mattress comprising an apparatus according to any of clauses 1-15.

1. A mattress having a substantially rectangular volume created by a stack of multiple parallel layers comprising a carrier layer consisting of an empty space in which a plurality of supporting springs are arranged, wherein the mattress comprises an antimicrobial air ventilation unit for treating air with UVC light, the antimicrobial air ventilation unit being arranged within the layer with the plurality of supporting springs in the volume of the mattress and comprising:
at least one centrifugal fan unit comprising an impeller and a ducted fan housing; and
at least one UVC radiation unit comprising (i) at least one UVC-LED and optionally (ii) at least one UVC directing lens,
wherein the UVC radiation unit(s) are configured to allow irradiation of air passing through the antimicrobial air ventilation unit,
wherein the at least one UVC radiation unit is configured to illuminate at least a part of the interior of the ducted fan housing with UVC light.

2. The mattress according to claim 1, wherein the at least one centrifugal fan unit is a cross-flow tangential centrifugal fan unit.

3. The mattress according to claim 1, wherein the impeller is a cylindrical impeller, or a hollow cylindrical impeller.

4. The mattress according to claim 1, wherein the impeller is a hollow cylindrical impeller.

5. The mattress according to claim 4, wherein the at least one centrifugal fan unit is a tangential centrifugal fan unit, the impeller is a hollow cylindrical impeller and the ends of the hollow cylindrical impeller are capped with solid end walls.

6. The mattress according to claim 1, wherein the UVC radiation units or the UVC LEDs are capable of producing light with one or more wavelengths in the range of from 100 to 280 nm.

7. The mattress according to claim 1, wherein the UVC-LED(s) have a maximum irradiance flux density of from 0.5 to 50 mW/cm² at a distance of 50 mm from the UVC-LED.

8. The mattress according to claim 1, wherein the UVC-LED(s) have a directivity angle 2q1/2 of from 180° to 5°.

9. The mattress according to claim 1, wherein the UVC radiation unit is configured to illuminate the interior of the ducted fan housing with UVC light, such that mechanical axis of the UVC-LED(s) is substantially co-axial with the axis of rotation of the impeller, wherein the axes are within 45° of co-axial.

10. The mattress according to claim 1, wherein the antimicrobial air ventilation unit comprises:
at least two UVC radiation units, each UVC radiation unit comprising at least one UVC-LED;
wherein the at least two UVC radiation units are configured to illuminate the interior of the ducted fan housing with UVC light, such that mechanical axis of the UVC-LED(s) intersects the ducted fan housing.

11. The mattress according to claim 1, wherein the antimicrobial air ventilation unit additionally comprises a light occluding baffle assembly.

12. The mattress according to claim 1, wherein the antimicrobial air ventilation unit for treating air with UVC light comprises:
at least one UVC radiation unit comprising at least one UVC-LED; and
at least one mirrored interior surface of the ducted fan housing
wherein the at least one UVC radiation unit is configured to illuminate the interior of the ducted fan housing with UVC light, such that the mechanical axis of the UVC-LED(s) intersects the mirrored interior surface of the ducted fan housing.

13. The mattress according to claim 1, wherein the UVC radiation unit is configured to illuminate a space defined by (i) the ducted fan housing, in particular in the space “down-wind” from the impeller when the antimicrobial air ventilation unit is in use.

14. The mattress according to claim 1, wherein the antimicrobial air ventilation unit is configured for drawing in air from either an intake opening in the mattress at a first surface or through a permeable lower support layer, with the drawn in air entering the air ventilation unit.

15. The mattress according to claim 14, wherein the multiple parallel layers in the mattress are each configured with a respective predetermined air permeability for controlling a flow of the air from the air ventilation unit thorough the mattress such that air expelled from the air ventilation unit permeates the layers depending on their respective permeability and substantially exits the mattress evenly distributed from a second surface, opposite the first surface.

16. A method for the antimicrobial treatment of an air flow in a mattress comprising the steps of:
i. Providing air comprising microbes to an antimicrobial air ventilation unit in the mattress according to claim 1;
ii. Causing the air comprising microbes to flow through the centrifugal fan unit;
iii. Irradiating the air comprising microbes with UVC light flowing through the centrifugal fan unit within the ducted fan housing of the antimicrobial air ventilation unit.
